SEQUENCE LISTING (2 sequences)

SEQ ID NO: 1 - DNA, 22 bp, Artificial Sequence
  synthetic DNA
tgactgtgaacgttcgagatga

SEQ ID NO: 2 - DNA, 22 bp, Artificial Sequence
  synthetic DNA
tgactgtgaaggttagagatga

(19) United States
(12) Patent Application Publication
(10) Pub. No.: US 2005/0180962 A1
(43) Pub. Date: Aug. 18, 2005

(54) Inactivated probiotic bacteria and methods of use thereof

(76) Inventors: Eyal Raz, Del Mar, CA (US); Daniel Rachmilewitz, Tel Aviv (IL)

Correspondence Address: BOZICEVIC, FIELD & FRANCIS LLP;Suite 200, 200 Middlefield Road, Menlo Park, CA, 94025, US

(21) Appl. No.: 10/742,052

(22) Filed: Dec. 18, 2003

Related U.S. Application Data
(60) Provisional application No. 60/443,922, filed on Jan. 30, 2003.

(51) Int. Cl. A61K 45/00; A61K 31/7012; A61K 9/48; A61K 9/20; A61K 31/715

(52) U.S. Cl. 424/93.45; 514/53; 514/58; 424/451; 424/464

(57) ABSTRACT

The present invention provides formulations comprising inactivated probiotic bacteria, and treatment methods using the formulations.

### CROSS-REFERENCE

This application claims the benefit of U.S. Provisional Patent Application No. 60/443,922 filed Jan. 30, 2003, which application is incorporated herein by reference in its entirety.

### STATEMENT REGARDING FEDERALLY SPONSORED RESEARCH

The U.S. government may have certain rights in this invention, pursuant to grant nos. AI40682 and DK-35108 awarded by the National Institutes of Health.

### FIELD OF THE INVENTION

The present invention is in the field of probiotics.

### BACKGROUND OF THE INVENTION

Probiotics are live microorganisms that alter the enteric microflora and have a beneficial effect on health. Probiotic formulations have been used as dietary supplements for many years. Resident probiotic bacterial strains live and reproduce in each person's digestive tract. Transient probiotic bacterial strains typically are introduced into the body through ingested food or by means of dietary supplements. Formulations of probiotic bacteria have been used to treat various gastrointestinal disorders, such as gastric ulcers, inflammatory bowel disease, acidic gut syndrome, gastritis, food allergies, and lactose intolerance. The use of probiotic bacteria to attenuate allergic disorders is also practiced.

There is a need in the art for formulations of probiotic bacteria that are storage stable and that can survive elevated temperatures. The present invention addresses this need by providing formulations comprising inactivated probiotic bacteria, which formulations are useful in a variety of treatment methods.

Literature

U.S. Pat. Nos. 6,461,607, 6,426,099, 6,468,525, 6,329,002, 6,203,797, 6,060,050, 6,264,952, 6,100,388, 5,922,375, 5,824,538; Pereyra et al. (1991) Eur. Cytokine Netw. 2:299-303; WO 01/62207; Schultz et al. (2002) Inflammatory Bowel Diseases 8:71-80; Steidler (2001) Microbes and Infection 3:1157-1166; Isolauri et al. (2002) Gut 50 (Suppl. III): iii54-iii59; Salminen et al. (2002) Clin. Infect. Dis. 35:1155-1160; Shanahan (2001) Gut 48:609; Kaur et al. (2002) Eur. J. Pharm. Sci. 15:1-9; Marteau et al. (2001) Am. J. Clin. Nutr. 73(suppl.):430S-436S; Teitelbaum and Walker (2002) Ann. Rev. Nutr. 22:107-138; Rachmilewitz et al. (2002) Gastroenterology 122:1428-1441; Kaila et al. (1992). Pediatr Res 32:141-144; Saavedra et al. (1994) Lancet 344:1046-1049; Corthier (1997) In: Probiotics 2. Applications and Practical Aspects (Fuller R, ed.). Chapman & Hal, London pp. 40-64; Gorbach et al. (1987) Lancet 2:1519; Goldin and Gorbach “Probiotics for humans.” In: Probiotics. The Scientific Basis (Fuller, ed) Chapman & Hall; London: 1992; 355-376; Biller et al. (1995) J Pediatr Gastroenterol Nutr 21: 224-226; Colombel et al. (1987) Lancet 1:43; Corthier et al. (1985) Appl Environ Microbiol 49:250-252; Banerjee and Lamont (2000) Curr Op Infect Dis 13:215-219; D'Souza et al. (2002) Brit Med J 324:1361; Gionchetti et al. (2000) J Gastroenterol Hepatol 15:489-493; Venturi et al. (1999) Ailment Pharmacol Ther 13:1103-1108; Gionchetti et al. (2000) Gastroenterology 119:305-309; Kruis et al. (1997) Aliment Pharmacol Ther 11:835-8; Bazzocohi et al. (1998) Gastroenterol Intern 11:111; Kim et al. (2003) Aliment Pharmacology & Therapeutics 17:895; De Simone et al. (1995) Microecol Ther 25:32-36; Delia et al. (2002) Am J. Gastroenterol 97:2150; Canducci et al. (2000) Aliment Pharmocol Ther 14:1625; Li et al. (2003) Hepatology 37:343; Macfarlane and Manzel (1998) J Immunol 160:1122-1131; Majamaa and Isolauri (1997) J Allergy Clin Immunol 99:179; Isolauri et al. (2000) Clin Exp Allergy 30:1604; Kalliomaki et al. (2001) Lancet 357:1076; Helin et al. (2002) Allergy 37:243; Wheeler et al. (1997) Ann Allergy Asthma Immunol 79:229; Matricardi et al. (2003) Allergy 58:461; Sudo et al. (1997) J Immunol 157:1739-1745.

### SUMMARY OF THE INVENTION

The present invention provides formulations comprising inactivated probiotic bacteria, and treatment methods using the formulations.

Features of the Invention

The present invention features an enteral formulation that includes at least about 5% by weight inactivated probiotic bacteria; and a pharmaceutically acceptable excipient. The bacteria are inactivated by a process other than extreme heat inactivation or bacteriophage infection. In some embodiments, the formulation is a liquid or gel formulation and includes an agent selected from a flavoring agent and a coloring agent. In other embodiments, the formulation is a solid formulation includes a solid-based dry material. In some embodiments in which the formulation is a solid formulation, the solid-based dry material is selected from a starch, gelatin, sucrose, dextrose, trehalose, and malto-dextrin. A subject formulation may be in the form of a capsule, tablet, a liquid, a gel, or a food product.

In some embodiments, the pharmaceutically acceptable excipient is a food-grade carrier. In some embodiments in which the pharmaceutically acceptable excipient is a food-grade carrier, the food-grade carrier is one or more of a carrier selected from an edible oil (e.g., olive oil), an emulsifier, a soluble fiber, a flavoring agent, a coloring agent, an edible fiber, and a sweetener.

In some embodiments, the inactivated bacteria are present in the formulation at a concentration of from about 1×10⁵ bacteria per dosage unit to about 1×10¹⁴ bacteria per dosage unit, e.g., per gram, per ml, per tablet, per packet, per capsule, per lozenge, or per serving size.

The present invention features a food product comprising subject inactivated bacteria. In some embodiments, the inactivated bacteria are present in the food product at a concentration of from about 1×10⁵ bacteria per dosage unit to about 1×10¹⁴ bacteria per dosage unit, e.g., per gram, per ml, per tablet, per packet, per capsule, per lozenge, or per serving size. In some embodiments, the food product is a milk-based food product, e.g., milk, cheese, yogurt, butter, ice cream, frozen yogurt, whipped toppings, cream, custard, pudding, nutritional drinks, infant formula, and milk chocolate. In other embodiments, the food product is a soy-based food product. In other embodiments, the food product is a starch-based food product. In other embodiments, the food product is a grain-based food product.

In some embodiments, the formulation further includes a non-steroidal anti-inflammatory agent. In other embodiments, the formulation further includes an antibiotic. In some embodiments, the formulation further includes an immunosuppressive agent. In other embodiments, the formulation further includes at least a second therapeutic agent for the treatment of a gastrointestinal disorder. In other embodiments, the formulation further includes at least a second therapeutic agent for the treatment of an allergic disorder.

The invention further features a method for treating gastrointestinal inflammation in a subject. The method generally involves administering to a subject suffering from gastrointestinal inflammation an effective amount of a subject formulation. In some embodiments, the formulation is administered orally. In other embodiments, the formulation is administered rectally. In some embodiments, the gastrointestinal inflammation is chronic gastrointestinal inflammation. In some embodiments, the chronic gastrointestinal inflammation is caused by inflammatory bowel disease. In some embodiments, the inflammatory bowel disease is ulcerative colitis. In other embodiments, the inflammatory bowel disease is Crohn disease. In many embodiments, from about 1×10⁵ bacteria per gram to about 1×10¹⁴ bacteria per unit dosage form are administered. In some embodiments, the method further involves administering an additional therapeutic agent for treating gastrointestinal inflammation. In other embodiments, the formulation further includes at least a second therapeutic agent for the treatment of diarrhea.

The present invention further features a method for treating an allergic disorder. The method generally involves administering to a subject suffering from an allergic disorder an effective amount of a subject formulation. In some embodiments, the formulation is administered orally. In many embodiments, from about 1×10⁵ bacteria per gram to about 1×10¹⁴ bacteria per unit dosage form are administered. In some embodiments, the method further involves administering an additional therapeutic agent for treating the allergic disorder. In some embodiments, the allergic disorder is allergic asthma. In some embodiments, the allergic disorder is an allergic reaction to a plant allergen, a food allergen, an animal allergen, or a drug allergen. In some embodiments, the allergic disorder is selected from atopic dermatitis, a food allergy, allergic asthma, allergic gastroenteritis, and allergic rhinitis.

The present invention further features a method of treating a diarrheal disease in an individual in need thereof. The method generally involves administering to an individual suffering from a diarrheal disease an effective amount of a subject formulation. In some embodiments, the formulation is administered orally. In many embodiments, from about 1×10⁵ bacteria per gram to about 1×10¹⁴ bacteria per unit dosage form are administered. Diarrheal diseases that are amenable to treatment with a subject formulation include diarrhea that results from a bacterial infection, a viral infection, or a mixed bacterial and viral infection; radiation-induced diarrhea; and antibiotic-induced diarrhea.

The present invention further features a method of treating a microbial infection in an individual. The method generally involves administering to an individual suffering from infection with a pathogenic microorganism an effective amount of a subject formulation. In some embodiments, the formulation is administered orally. In many embodiments, from about 1×10⁵ bacteria per gram to about 1×10¹⁴ bacteria per unit dosage form are administered. In some embodiments, the pathogenic microorganism is one that gives rise to an opportunistic infection, e.g., in an immunodeficient individual. In other embodiments, the microorganism is one that gives rise to or is associated with gastric ulcers, e.g., Helicobacter pylori.

The present invention further features a method for treating a non-alcoholic fatty liver disease in an individual. The method generally involves administering to a subject suffering from non-alcoholic fatty liver disease an effective amount of a subject formulation. In some embodiments, the formulation is administered orally. In many embodiments, from about 1×10⁵ bacteria per gram to about 1×10¹⁴ bacteria per unit dosage form are administered. In some embodiments, the non-alcoholic liver disease is steatosis. In other embodiments, the non-alcoholic liver disease is non-alcoholic steatohepatitis. Non-alcoholic fatty liver disease frequently results in fibrosis or cirrhosis. The subject methods for treating non-alcoholic fatty liver disease reduce the risk that an individual suffering from non-alcoholic fatty liver disease will develop fibrosis or cirrhosis of the liver. Thus, the invention further features a method for reducing the risk that an individual will develop hepatic fibrosis or cirrhosis.

### BRIEF DESCRIPTION OF THE DRAWINGS

FIGS. 1A-C depict immunostimulatory activities of probiotic DNA.

FIGS. 2A-C depict detection of bacterial DNA at systemic sites.

### DEFINITIONS

As used herein, the term “inactivated probiotic bacteria” refers to probiotic bacteria that are inactivated in a manner such that the bacteria retain a beneficial effect in treating a disorder in an individual, e.g., a gastrointestinal inflammatory disorder and/or an allergic disorder and/or a microbial infection. Viable probiotic bacteria are typically found in the gastrointestinal tract as part of the normal flora in healthy individuals. Probiotic bacteria for use in a subject formulation are generally non-pathogenic and non-toxigenic when viable, e.g., bacteria suitable for use herein are non-pathogenic and non-toxic even before inactivation. Inactivated probiotic bacteria of the instant invention typically do not elicit an immune response to an antigen of the probiotic bacteria, and typically do not elicit an immune response that provides protection against the probiotic bacteria. Inactivated probiotic bacteria of the instant invention generally comprise nucleic acid that is capable of stimulating a Th1-type immune response in an individual.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) reducing the incidence and/or risk of relapse (remission, “flare-up”) of the disease during a symptom-free period; (b) relieving or reducing a symptom of the disease; (c) preventing the disease from occurring in a subject which may be predisposed to the disease but has not yet been diagnosed as having it; (d) inhibiting the disease, i.e., arresting its development (e.g., reducing the rate of disease progression); (e) reducing the frequency of episodes of the disease; and (f) relieving the disease, i.e., causing regression of the disease.

The terms “individual,” “host,” “subject,” and “patient,” used interchangeably herein, refer to a mammal, particularly a human.

“Gastrointestinal inflammation” as used herein refers to inflammation of a mucosal layer or all of the layers of the gastrointestinal tract, and encompasses acute and chronic inflammatory conditions. Acute inflammation is generally characterized by a short time of onset and infiltration or influx of neutrophils. Chronic inflammation is generally characterized by a relatively longer period of onset and infiltration or influx of mononuclear cells. Chronic inflammation can also typically characterized by periods of spontaneous remission and spontaneous occurrence. “Mucosal layer of the gastrointestinal tract” is meant to include mucosa of the bowel (including the small intestine and large intestine), rectum, stomach (gastric) lining, oral cavity, and the like. Certain gastrointestinal disorders affect all layers of the gastrointestinal tract. For example, Crohn disease is known to involve all layers of the gastrointestinal tract, including the mucosal layer, the muscle layer, and the serosal layer.

“Chronic gastrointestinal inflammation” refers to inflammation of the mucosa of the gastrointestinal tract that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic gastrointestinal inflammation may be expected to require a long period of supervision, observation, or care. “Chronic gastrointestinal inflammatory conditions” (also referred to as “chronic gastrointestinal inflammatory diseases”) having such chronic inflammation include, but are not necessarily limited to, inflammatory bowel disease (IBD), colitis induced by environmental insults (e.g., gastrointestinal inflammation (e.g., colitis) caused by or associated with (e.g., as a side effect) a therapeutic regimen, such as administration of non-steroidal anti-inflammatory drugs (NSAIDS), chemotherapy, radiation therapy, and the like), colitis in conditions such as chronic granulomatous disease (Schappi et al. Arch Dis Child. 2001 February; 84(2):147-151), celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammation caused by an infectious agent, and other like conditions.

As used herein, “inflammatory bowel disease” or “IBD” refers to any of a variety of diseases characterized by inflammation of all or part of the intestines. Examples of inflammatory bowel disease include, but are not limited to, Crohn disease and ulcerative colitis. Reference to IBD in this specification is often referred to in the specification as exemplary of gastrointestinal inflammatory conditions, and is not meant to be limiting.

The term “allergic disorder” generally refers to a disease state or syndrome whereby the body produces an immune response to environmental antigens comprising immunoglobulin E (IgE) antibodies which evoke allergic symptoms such as itching, sneezing, coughing, respiratory congestion, rhinorrhea, skin eruptions and the like, as well as severe reactions, such as asthma attacks and systemic anaphylaxis. Examples of allergic diseases and disorders which can be treated by the methods of this invention include, but are not limited to, drug hypersensitivity, allergic rhinitis, bronchial asthma, ragweed pollen hayfever, anaphylactic syndrome, urticaria, angioedema, atopic dermatitis, erythema nodosum, erythema multiforme, Stevens Johnson Syndrome, cutaneous necrotizing venulitis, bullous skin diseases, allergy to food substances and insect venom-induced allergic reactions, as well as any other allergic disease or disorder.

The terms “CD4⁺-deficient” and “CD4⁺-low” are used interchangeably herein, and, as used herein, refer to a state of an individual in whom the number of CD4⁺ T lymphocytes is reduced compared to an individual with a healthy, intact immune system. CD4⁺ deficiency includes a state in which the number of functional CD4⁺ T lymphocytes is less than about 600 CD4⁺ T cells/mm³ blood; a state in which the number of functional CD4⁺ T cells is reduced compared to a healthy, normal state for a given individual; and a state in which functional CD4⁺ T cells are completely absent.

As used herein, a “CD4⁺-deficient individual” is one who has a reduced number of functional CD4⁺-T cells, regardless of the reason, when compared to an individual having a normal, intact immune system. In general, the number of functional CD4⁺-T cells that is within a normal range is known for various mammalian species. In human blood, e.g., the number of functional CD4⁺-T cells which is considered to be in a normal range is from about 600 to about 1500 CD4⁺-T cells/mm³ blood. An individual having a number of CD4⁺-T cells below the normal range, e.g., below about 600/mm³, may be considered “CD4⁺-deficient.” Thus, a CD4⁺-deficient individual may have a low CD4⁺ T cell count, or even no detectable CD4⁺ T cells. A CD4⁺-deficient individual includes an individual who has a lower than normal number of functional CD4⁺-T cells due to a primary or an acquired immunodeficiency.

A “functional CD4⁺-T cell” is a term well understood in the art and refers to a CD4⁺-T cell which is capable of providing T cell help, directly or indirectly, to effect one or more of the following responses: CTL activation; antibody production; macrophage activation; mast cell growth; and eosinophil growth and differentiation.

As used herein, the terms “immunodeficient,” “immunosuppressed,” and “immunocompromised,” used interchangeably herein, refer to a state of a CD4⁺-deficient individual.

As used herein, “pharmaceutically acceptable carrier” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed. or latest edition, Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a probiotic bacterium” includes a plurality of such bacteria and reference to “the dosage unit” includes reference to one or more dosage units and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides formulations comprising inactivated probiotic bacteria, and various therapeutic methods using the formulations. Subject formulations are useful for treating any disorder that is amenable to treatment with viable probiotic bacteria. Thus, the present invention provides methods of treating gastrointestinal inflammation; microbial infections; diarrheal diseases; allergic disorders; non-alcoholic liver disease; and the like.

The invention is based on the observation that irradiated probiotic bacteria, but not probiotic bacteria inactivated by extreme heat, are efficacious in treating colitis in an animal model of colitis. Thus, contrary to what has been reported in the art, probiotic bacteria need not be alive to exert a beneficial effect. Without wishing to be bound by theory, the reason that irradiate probiotic bacteria are effective, while bacteria killed by treatment at 100° C. for 30 minutes are not, may relate to maintenance of structural integrity (e.g., of the cell wall and/or cytosolic components) in the former, but not in the latter, bactericidal method.

Currently available probiotic formulations are typically stored at a temperature of no higher than 45° C., and usually are either lyophilized, or are stored in an aqueous or other non-frozen medium at refrigeration temperatures (e.g., at about 4° C.), because at higher temperatures, viability of probiotic bacteria is reduced. The formulations of the present invention are advantageous over currently available probiotic formulations in that they need not be maintained within a particular temperature range, as required of live probiotic cultures. The subject probiotic formulations are storage stable over a wide temperature range.

A further advantage of the formulations of the instant invention lies in the fact that, because the inactivated probiotic bacteria formulations of the invention are non-viable or have reduced viability, they can be administered safely to immunocompromised individuals, and to infants.

Probiotic Formulations

The present invention provides formulations comprising inactivated probiotic bacteria, and methods of making the formulations. The formulations are suitable for human consumption, and may include one or more pharmaceutical excipients, including food-grade excipients. As such, the subject invention further provides food products that include inactivated probiotic bacteria.

Probiotic bacteria included in the formulations of the present invention are non-pathogenic and non-toxigenic when viable. Such bacteria may be found as part of the bacterial flora of the normal, healthy human intestine. In many embodiments, inactivated probiotic bacteria included in the formulations of the present invention are or have been isolated from their natural environment, e.g., or are variants of bacteria isolated from their natural environment. A number of probiotic bacteria are commercially available. Variants include bacteria with naturally-occurring mutations; bacteria that have been manipulated in the laboratory to differ genetically from a naturally occurring bacteria (e.g., by the introduction of one or more mutations, by the introduction of exogenous polynucleotides (e.g., “recombinant” or genetically modified bacteria, etc.). Typically, subject bacteria are grown in in vitro culture before being inactivated.

Suitable bacteria for inclusion in the instant formulations include, but are not limited to, bacteria of various species, including lactobacillus species, e.g., Lactobacillus acidophilus, L. plantarum, L. casei, L. rhamnosus, L. delbrueckii (including subspecies bulgaricus), L. reuteri, L. fermentum, L. brevis, L. lactis, L. cellobiosus, L. GG, L. gasseri, L. johnsonii, and L. plantarum; bifidobacterium species, e.g., Bifidobacterium bifidum, B. infantis, B. longum, B. thermophilum, B. adolescentis, B. breve, B. animalis; streptococcus species, e.g., Streptococcus lactis, S. cremoris, S. salivarius (including subspecies thermophilus), and S. intermedius; Leuconostoc species; Pediococcus species; Propionibacterium species; Bacillus species; non-enteropathogenic Escherichia species, e.g., non-enteropathogenic Escherichia coli, e.g., E. coli Nissle, and the like; and Enterococcus species such as Enterococcus faecalis, and E. faecium. Other suitable probiotic bacteria are known in the art, and have been described. See, e.g., U.S. Pat. No. 5,922,375. The person skilled in the art would understand and recognize those microorganisms which may be included in the compositions of the invention.

Bacteria other than the bacteria that are commonly considered as probiotic bacteria can also be used in a subject formulation. For example, bacteria that are pathogenic when viable can also be used, since the bacteria are inactivated before use. Essentially any bacteria that is capable, when inactivated, of alleviating the symptoms of a disorder amenable to treatment with viable probiotic bacteria, e.g., a gastrointestinal inflammatory disorder, a microbial infection, an allergic disorder, and the like, can be included in a subject formulation.

In some embodiments, a subject formulation includes two or more different inactivated probiotic bacteria, e.g., the bacteria may differ in strain, species, or genus. The bacteria may differ in, e.g., strain, species, or genus. As one non-limiting example, a formulation comprises S. salivarius subsp. thermophilus, B. breve, B. infantis, B. longum, L. acidophilus, L. casei, and L. delbrueckii subsp. bulgaricus. For example, the combinations of bacteria found in a commercially available product such as Kyo-Dophilus capsules (Wakunaga Probiotics), Kyo-Dophilus tablets (Wakunaga Probiotics), Acidophilas® (Wakunaga Probiotics), Probiata® tablets, Flora Grow (Arise & Shine), Bifa 15 (Eden Foods), TH1 Probiotics (Jarrow Formulasa), Replenish (Innercleanse 2000), Flora BaC™ (PDI Labs), Subalin, Colinfant, Mutaflor, and the like.

As another non-limiting example, a subject probiotic formulation comprises two different Lactobacillus strains, e.g., different isolates of the same species that are genetically diverse. As another non-limiting example, a subject probiotic formulation comprises from one to four Lactobacillus strains and from one to four Bifidobacterium strains. As another non-limiting example, a subject probiotic formulation comprises from one to four lactobacillus strains, from one to four bifidobacterium strains, and a non-enteropathogenic E. coli strain. As another non-limiting example, a subject probiotic formulation comprises from one to four lactobacillus strains and a non-enteropathogenic E. coli strain. As another non-limiting example, a subject probiotic formulation comprises from one to four bifidobacterium strains, and a non-enteropathogenic E. coli strain.

The probiotic bacteria in the subject formulations are inactivated. As used herein, the term “inactivated” refers to non-viable bacteria or bacteria with reduced viability. The probiotic bacteria of the subject formulations are inactivated such that bacterial growth in vitro is inhibited. In many embodiments, inactivated bacteria are unable to grow in in vitro culture, e.g., growth in in vitro culture is undetectable. Whether bacterial growth is inhibited in vitro can be determined using well-known methods, e.g., plating the bacteria on agar supplemented with suitable growth medium (e.g., Luria-Bertani broth, DeMan, Rogosa, Sharpe (MRS) broth, and the like); and counting the number of colonies formed after overnight (e.g., 12-16 hours) culture at 37° C. The number of colony-forming units (cfu) is a measure of the viability of the culture. Bacterial growth in vitro can also be determined by culturing the bacteria in liquid medium containing appropriate nutritional supplements, and, after a period of about 12-16 hours at 37° C., the turbidity of the culture medium is measured, e.g., absorbance at, e.g., 570-600 nm.

The probiotic bacteria of the subject formulations are inactivated by a process other than extreme heat inactivation, e.g., the inactivated probiotic bacteria are not inactivated by heating to 100° C. for 30 minutes. Subject inactivated bacteria are inactivated in such a manner such that they cannot replicate, and in such a manner that allows for the release of DNA from the bacteria following introduction into the gastrointestinal tract of an individual. The probiotic bacteria of the subject formulations are not inactivated by infection with bacteriophage. Inactivation can be achieved by various processes other than heat inactivation, including, but not limited to, irradiation; treatment with microwaves (e.g., treatment with 915 MHz or 2450 MHz); treatment with radio frequencies; treatment with antibiotics; pasteurization; and treatment with chemical agents that reduce viability. In many embodiments, the inactivated bacteria remain intact, e.g., the cell wall of the bacteria remains relatively intact following the inactivation procedure.

In other embodiments, the cell wall does not remain intact following the inactivation procedure. In these embodiments, the inactivation process may result in holes in the cell wall, or may result in partial or complete breakdown of the cell wall. Disruption of the integrity of the cell wall may occur following certain inactivation procedures, such as freeze-thaw inactivation; and the like.

Chemical agents include, but are not limited to, aldehydes, e.g., formaldehyde, glutaraldehyde, and the like; food preservative agents such as SO₂, sorbic acid, benzoic, acid, nitrate, and nitrite salts; gases such as ethylene oxide; halogens, such as iodine, chlorine, and the like; peroxygens, such as ozone, peroxide, peracetic acid; bisphenols; phenols; phenolics; biguanides, e.g., chlorhexidine; and the like.

Antibiotics include, but are not limited to, Gentamicin; Vancomycin; Oxacillin; Tetracyclines; Nitroflurantoin; Chloramphenicol; Clindamycin; Trimethoprimsulfamethoxasole; a member of the Cephlosporin antibiotic family (e.g., Cefaclor, Cefadroxil, Cefixime, Cefprozil, Ceftriaxone, Cefuroxime, Cephalexin, Loracarbef, and the like); a member of the Penicillin family of antibiotics (e.g., Ampicillin, Amoxicillin/Clavulanate, Bacampicillin, Cloxicillin, Penicillin VK, and the like); with a member of the Fluoroquinolone family of antibiotics (e.g., Ciprofloxacin, Grepafloxacin, Levofloxacin, Lomefloxacin, Norfloxacin, Ofloxacin, Sparfloxacin, Trovafloxacin, and the like); or a member of the Macrolide antibiotic family (e.g., Azithromycin, Erythromycin, and the like); and neomycin.

In some embodiments, the probiotic bacteria are irradiated. The probiotic bacteria are irradiated at an energy and for a period of time sufficient to inhibit bacterial growth in vitro and/or to render the probiotic bacteria non-viable, e.g., such that growth in in vitro culture is undetectable using standard methods. In some embodiments, the irradiation is ionizing radiation. Gamma radiation is an example of ionizing radiation. For example, the bacteria are irradiated using gamma irradiation in an amount of from about 5 kiloGray (kGy) to about 50 kGy, from about 10 kGy to about 20 kGy, from about 20 kGy to about 40 kGy, or from about 25 kGy to about 35 kGy. Bacteria are irradiated for a period of time of from about 15 seconds to about 48 hours, e.g., from about 15 seconds to about 1 minute, from about 1 minute to about 15 minutes, from about 15 minutes to about 30 minutes, from about 60 minutes to about 90 minutes, from about 90 minutes to about 2 hours, from about 2 hours to about 4 hours, from about 4 hours to about 8 hours, from about 8 hours to about 12 hours, from about 12 hours to about 16 hours, from about 16 hours to about 24 hours, from about 24 hours to about 36 hours, or from about 36 hours to about 48 hours. The total amount of irradiation and the duration of irradiation can be adjusted, depending on various factors, e.g., the number of bacteria being irradiated. The total amount of irradiation and the duration of irradiation that results in bacteria that have reduced viability or are non-viable (e.g., are unable to grow in in vitro culture) are readily determined by those of ordinary skill in the art.

In other embodiments, the radiation is ultraviolet (UV) radiation. For example, the probiotic bacteria are exposed to UV radiation of from about 2000 μW sec/cm² to about 1,000 μW sec/cm².

In some embodiments, the probiotic bacteria are inactivated by pasteurization. The process of pasteurization is well known in the art of food sciences. Any method for pasteurization can be used for the current invention. Pasteurization generally involves heating the material to be pasteurized at one of the following temperatures, for the following time period: at about 60° C. for at least about 30 minutes; at 72° C. for at least about 15 seconds; at 88° C. for at least about 1 second; at 90° C. for at least about 0.5 second; at 94° C. for about 0.1 second; at 96° C. for about 0.05 second; or 100° C. for about 0.01 second. Standard pasteurization conditions are found in the literature, e.g., in U.S. Pat. Nos. 6,475,545, 4,438,147, and 6,528,085. For example, in the present invention, pasteurization of liquids or solids comprising a suitable probiotic bacterium is carried out by heating the liquid or solid under conventional pasteurization conditions such as, for example, but not limited to, about 72° C. to about 85° C. for from about 15 seconds to about 10 minutes, e.g., about 72° C. to about 85° C. for from about 15 seconds to about 30 seconds, from about 20 seconds to about 40 seconds, from about 30 seconds to about 60 seconds, from about 1 minute to about 2 minutes, from about 2 minutes to about 5 minutes, or from about 5 minutes to about 10 minutes. Generally, temperatures above 90° C. are not used to inactivate bacteria in the present invention.

Viability is reduced by at least about 50%, at least about 60%, at least about 70%, at least about 80%, at least about 90%, at least about 95%, or at least about 99%, or more, such that fewer than about 50%, fewer than about 40%, fewer than about 30%, fewer than about 20%, fewer than about 10%, fewer than about 5%, or fewer than about 1%, or fewer, of the bacteria in the formulation are viable. In some embodiments, 100% of the bacteria are non-viable, e.g., are unable to grow in in vitro culture.

Viability of bacteria is determined using any known method. For example, bacteria are contacted with a membrane-permeant fluorescent dye (e.g., SYTO 9, SYTOX, and the like) that labels live bacteria with green fluorescence; and membrane-impermeant propidium iodide that labels membrane-compromised bacteria with red fluorescence. Roth et al. (1997) Appl. Environ. Microbiol. 63:2421-2431; Lebaron et al. (1998) Appl. Environ. Microbiol. 64:2697-2700; and Braga et al. (2003) Antimicrob. Agents Chemother. 47:408-412. Bacterial viability is also determined by plating the bacteria on an agar plate containing requisite nutritional supplements, and counting the number of colonies formed (colony forming units, cfu).

Inactivated probiotic bacteria of the invention are stable at temperatures from about 10° C. to about 80° C., from about 15° C. to about 75° C., from about 20° C. to about 70° C., from about 25° C. to about 65° C., from about 30° C. to about 60° C., or from about 35° C. to about 55° C. For example, inactivated probiotic bacteria of the invention are stable at temperatures from about 10° C. to about 60° C., from about 20° C. to about 60° C., or from about 30° C. to about 60° C. In many embodiments, the inactivated probiotic bacteria are storage stable for a period of weeks, months, or years at the indicated temperature ranges.

A subject formulation comprises from about 5% to about 90%, from about 10% to about 85%, from about 15% to about 80%, from about 20% to about 75%, from about 25% to about 70%, from about 30% to about 65%, or from about 35% to about 60%, by weight or by volume, inactivated probiotic bacteria.

Formulations according to the present invention are prepared so that a liquid unit form contains from about 1×10⁵ to about 1×10¹⁴, from about 5×10⁵ to about 5×10¹³, from about 1×10⁶ to about 1×10¹², from about 5×10⁶ to about 5×10¹¹, or from about 1×10⁷ to about 1×10¹⁰ bacteria per unit dosage form, e.g., per ml, per gram, per tablet, per capsule, per packet, per serving size, etc. Formulations according to the present invention are prepared so that a solid, semi-solid, or gel unit form contains from about 1×10⁵ to about 1×10¹⁴, from about 5×10⁵ to about 5×10¹³, from about 1×10⁶ to about 1×10¹², from about 5×10⁶ to about 5×10¹¹, or from about 1×10⁷ to about 1×10¹⁰ bacteria per unit dosage form, e.g., per gram, per tablet, per packet, per capsule, per serving size, etc.

The following are non-limiting examples of unit dosage forms of a subject formulation: 1-5×10¹⁰ inactivated bacteria per packet, tablet, or capsule; 1-5×10¹¹ inactivated bacteria per packet, tablet, or capsule; 1-5×10¹² inactivated bacteria per packet, tablet, or capsule; 1-5×10¹³ inactivated bacteria per packet, tablet, or capsule; 1-5×10¹⁴ inactivated bacteria per packet, tablet, or capsule; 1-5×10¹⁰ inactivated bacteria per ml liquid formulation; 1-5×10¹¹ inactivated bacteria per ml liquid formulation; 1-5×10¹² inactivated bacteria per ml liquid formulation; 1-5×10¹³ inactivated bacteria per ml liquid formulation; 1-5×10¹⁴ inactivated bacteria per ml liquid formulation. The unit dosage forms can be packaged in multiples, e.g., the formulation is provided in a package of 4, 8, 12, 16, or 20 unit dosage forms.

The term “unit dosage form,” as used herein, refers to physically discrete units suitable as unitary dosages for human and animal subjects, each unit containing a predetermined quantity of inactivated probiotic bacteria of the present invention calculated in an amount sufficient to produce the desired effect in association with a pharmaceutically acceptable diluent, carrier or vehicle. The specifications for the novel unit dosage forms of the present invention depend on the particular bacteria or combination of bacteria employed and the effect to be achieved. The instant formulations are typically provided in unit dosage forms. In such form, the formulation is subdivided into unit doses containing appropriate quantities of the inactivated probiotic bacteria. The unit dosage form can be a packaged preparation, the package containing discrete quantities of formulation, such as packeted tablets, capsules, and powders in vials or ampoules. Also, the unit dosage form can be a capsule, tablet, cachet, suppository, or lozenge itself, or it can be the appropriate number of any of these in packaged form. A “unit dosage form” may be in the form of a table or capsule; a unit amount of a liquid or gel formulation; or, where the formulation is in the form of a food product or a nutraceutical, a serving size.

Formulations

In general, inactivated probiotic bacteria are formulated in a pharmaceutically acceptable composition for delivery to a host. In some embodiments, inactivated probiotic bacteria are formulated with a pharmaceutically acceptable carrier suitable for a solid or semi-solid formulation. In some embodiments, inactivated probiotic bacteria are formulated with a pharmaceutically acceptable carrier suitable for a liquid or gel formulation.

Inactivated probiotic bacteria formulations of the invention are typically formulated for enteral delivery, e.g., oral delivery, or delivery as a suppository, but can also be formulated for parenteral delivery, e.g., vaginal delivery, inhalational delivery (including oral delivery, nasal delivery, and intrapulmonary delivery), and the like.

The inactivated probiotic bacteria of the present invention may be formulated in a wide variety of oral administration dosage forms, with one or more pharmaceutically acceptable carriers. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material. In powders, the carrier is a finely divided solid which is a mixture with the inactivated probiotic bacteria. In tablets, the inactivated bacteria are mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the inactivated probiotic bacteria, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations such as emulsions, syrups, elixirs, aqueous solutions, aqueous suspensions, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by mixing the inactivated probiotic bacteria with water and adding suitable colorants, flavors, stabilizing and thickening agents. Aqueous suspensions can be prepared by dispersing the inactivated probiotic bacteria in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

Exemplary pharmaceutically carriers include sterile aqueous of non-aqueous solutions, suspensions, and emulsions. Examples of non-aqueous solvents are propylene glycol, polyethylene glycol, vegetable or seed oils such as olive oil, and injectable organic esters such as ethyl oleate. Aqueous carriers include water, alcoholic/aqueous solutions, emulsions or suspensions, including saline and buffered media. A composition of inactivated probiotic bacteria may also be lyophilized using means well known in the art, for subsequent reconstitution and use according to the invention. Also of interest are formulations for liposomal delivery, and formulations comprising encapsulated or microencapsulated inactivated probiotic bacteria.

The formulations of the present invention may also include known antioxidants, buffering agents, and other agents such as coloring agents, flavorings, vitamins or minerals. For example, a subject formulation may also contain one or more of the following minerals: calcium citrate (15-350 mg); potassium gluconate (5-150 mg); magnesium citrate (5-15 mg); and chromium picollinate (5-200 μg). In addition, a variety of salts may be utilized, including calcium citrate, potassium gluconate, magnesium citrate and chromium picollinate. Thickening agents may be added to the compositions such as polyvinylpyrrolidone, polyethylene glycol or carboxymethylcellulose. Exemplary additional components of a subject formulation include assorted colorings or flavorings, vitamins, fiber, milk, fruit juices, enzymes and other nutrients. Exemplary sources of fiber include any of a variety of sources of fiber including, but not limited to: psyllium, rice bran, oat bran, corn bran, wheat bran, fruit fiber and the like. Dietary or supplementary enzymes such as lactase, amylase, glucanase, catalase, and the like can also be included. Chemicals used in the present compositions can be obtained from a variety of commercial sources, including, e.g., Spectrum Quality Products, Inc (Gardena, Calif.), Sigma Chemicals (St. Louis, Mo.), Seltzer Chemicals, Inc., (Carlsbad, Calif.) and Jarchem Industries, Inc., (Newark, N.J.).

A subject formulation may also include a variety of carriers and/or binders. An exemplary carrier is micro-crystalline cellulose (MCC) added in an amount sufficient to complete dosage total weight. Carriers can be solid-based dry materials for formulations in tablet, capsule or powdered form, and can be liquid or gel-based materials for formulations in liquid or gel forms, which forms depend, in part, upon the routes of administration.

Typical carriers for dry formulations include, but are not limited to: trehalose, malto-dextrin, rice flour, micro-crystalline cellulose (MCC) magnesium sterate, inositol, fructo-oligosaccharide (FOS), gluco-oligosaccharide (GOS), dextrose, sucrose, and like carriers. Where the composition is dry and includes evaporated oils that produce a tendency for the composition to cake (adherence of the component spores, salts, powders and oils), dry fillers which distribute the components and prevent caking are included. Exemplary anti-caking agents include MCC, talc, diatomaceous earth, amorphous silica and the like, and are typically added in an amount of from approximately 1% to 95% by weight. It should also be noted that dry formulations which are subsequently rehydrated (e.g., liquid formula) or given in the dry state (e.g., chewable wafers, pellets, capsules, or tablets) can be used instead of initially hydrated formulations. Dry formulations (e.g., powders) may be added to supplement commercially available foods (e.g., liquid formulas, strained foods, or drinking water supplies). Similarly, the specific type of formulation depends upon the route of administration.

Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Generally, water-based carriers possess a neutral pH value (e.g., pH 7.0±1.0 or 0.5 pH units). The compositions may also include natural or synthetic flavorings and food-quality coloring agents, all of which must be compatible with maintaining viability of the lactic acid-producing microorganism. Well-known thickening agents may also be added to the compositions such as corn starch, guar gum, xanthan gum, and the like.

Preservatives may also be included within the carrier including methylparaben, propylparaben, benzyl alcohol and ethylene diamine tetraacetate salts. Well-known flavorings and/or colorants may also be included within the carrier. The compositions of the present invention may also include a plasticizer such as glycerol or polyethylene glycol (e.g., in a molecular weight range of MW=800 to 20,000). The composition of the carrier can be varied so long as it does not interfere significantly with the pharmacological activity of the inactivated probiotic bacteria.

Inactivated probiotic bacteria can be formulated to be suitable for oral administration in a variety of ways, for example in a liquid, a powdered food supplement, a paste, a gel, a solid food, a packaged food, a wafer, a tablet, a lozenge, a capsule, and the like. Other formulations will be readily apparent to one skilled in the art.

In general, the pharmaceutical compositions can be prepared in various forms, such as granules, tablets, lozenges, pills, suppositories, capsules (e.g. adapted for oral delivery), microbeads, microspheres, liposomes, suspensions, and the like. The inactivated probiotic bacteria useful in the invention can be prepared in a variety of formulations, including conventional pharmaceutically acceptable carriers, and, for example.

Inactivated probiotic bacteria may be formulated with an inert diluent or with an assimilable edible carrier, or may be enclosed in hard or soft shell gelatin capsule, or may be compressed into tablets designed to pass through the stomach (i.e., enteric coated), or may be incorporated directly with the food of the diet. For oral therapeutic administration, the inactivated probiotic bacteria may be incorporated with excipients and used in the form of ingestible tablets, buccal tablets, troches, capsules, elixirs, suspensions, syrups, wafers, and the like.

The tablets, troches, pills, capsules, and the like, as described above, may also contain the following: a binder such as gum tragacanth, acacia, a starch (such as corn starch), or gelatin; excipients such as dicalcium phosphate; a disintegrating agent such as corn starch, potato starch, alginic acid, and the like; a lubricant such as magnesium stearate; and a sweetening agent such as sucrose, lactose or saccharin may be added or a flavoring agent such as peppermint, oil or wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, lozenges, pills or capsules or bacteria in suspension may be coated with shellac, sugar or both.

A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the inactivated probiotic bacteria may be incorporated into sustained-release preparations and formulations.

Pharmaceutical grade organic or inorganic carriers and/or diluents suitable for oral use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value, can also be added.

Some other examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; calcium carbonate; vegetable oils, such as peanut oils, cotton seed oil, sesame oil, olive oil, corn oil and oil of theobroma; polyols such as propylene glycol, glycerine, sorbitol, mannitol, and polyethylene glycol; agar; alginic acids; pyrogen-free water; isotonic saline; cranberry. extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tabletting agents, stabilizers, antioxidants and preservatives, can also be present.

A colloidal dispersion system may be used for oral delivery of inactivated probiotic bacteria. Colloidal dispersion systems include macromolecule complexes, nanocapsules, microspheres, beads, liposomes and the like.

The inactivated probiotic bacteria of the present invention may be formulated for administration as suppositories. A low melting wax, such as a mixture of fatty acid glycerides or cocoa butter is first melted and the inactivated probiotic bacteria are dispersed homogeneously, for example, by stirring. The molten homogeneous mixture is then poured into conveniently sized molds, allowed to cool, and to solidify.

The inactivated probiotic bacteria of the present invention may be formulated for vaginal administration. Pessaries, tampons, creams, gels, pastes, foams or sprays, may contain agents in addition to the bacteria, such carriers, known in the art to be appropriate.

In some embodiments, inactivated probiotic bacteria are formulated for delivery by inhalation. As used herein, the term “aerosol” is used in its conventional sense as referring to very fine liquid or solid particles carries by a propellant gas under pressure to a site of therapeutic application. The term “liquid formulation for delivery to respiratory tissue” and the like, as used herein, describe compositions comprising inactivated probiotic bacteria with a pharmaceutically acceptable carrier in flowable liquid form. Such formulations, when used for delivery to a respiratory tissue, are generally solutions, e.g. aqueous solutions, ethanolic solutions, aqueous/ethanolic solutions, saline solutions and colloidal suspensions.

In general, aerosolized particles for respiratory delivery must have a diameter of 12 microns or less. However, the preferred particle size varies with the site targeted (e.g, delivery targeted to the bronchi, bronchia, bronchioles, alveoli, or circulatory system). For example, topical lung treatment can be accomplished with particles having a diameter in the range of 1.0 to 12.0 microns. Effective systemic treatment requires particles having a smaller diameter, generally in the range of 0.5 to 6.0 microns. Thus, in some embodiments, at least about 40%, at least about 50%, at least about 60%, at least about 70%, at least about 80%, at least about 90%, or more, of an aerosolized formulation comprising inactivated probiotic bacteria for delivery to a respiratory tissue is composed of particles in the range of from about 0.5 to about 12 micrometers, from about 0.5 to about 6 micrometers, or from about 1.0 to about 12 micrometers.

The formulation for delivery to a respiratory tissue may be provided in a container suitable for delivery of aerosolized formulations. U.S. Pat. Nos. 5,544,646; 5,709,202; 5,497,763; 5,544,646; 5,718,222; 5,660,166; 5,823,178; 5,829,435; and 5,906,202 describe devices and methods useful in the generation of aerosols suitable for drug delivery, any of which can be used in the present invention for delivering a formulation comprising inactivated probiotic bacteria to a respiratory tissue. In some embodiments, the invention provides a container, which may be a disposable container, having at least one wall that is collapsible or movable upon application of a force, wherein at least one wall has an opening. A porous membrane having pores in a range of from about 0.25 microns to about 6 microns covers the opening. The container comprises a flowable liquid formulation comprising inactivated probiotic bacteria. Upon application of a force, the flowable liquid formulation is forced through the pores in the membrane and is aerosolized. The container may be provided in any known configuration, e.g., a blister pack. The container may be provided together with an aerosol delivery device, such that the aerosolized formulation exits the container and proceeds through a channel in an aerosol delivery device and into the respiratory tract of an individual.

When a pharmaceutical aerosol is employed in this invention, the aerosol contains inactivated probiotic bacteria, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Examples of suitable propellants include, but is not limited to, hydrocarbons or other suitable gas. In the case of the pressurized aerosol, the dosage unit may be determined by providing a value to deliver a metered amount.

Administration of formulation comprising inactivated probiotic bacteria can also be carried out with a nebulizer, which is an instrument that generates very fine liquid particles of substantially uniform size in a gas. Preferably, a liquid containing the inactivated probiotic bacteria is dispersed as droplets. The small droplets can be carried by a current of air through an outlet tube of the nebulizer. The resulting mist penetrates into the respiratory tract of the patient.

A powder composition containing inactivated probiotic bacteria, with or without a lubricant, carrier, or propellant, can be administered to a mammal in need of therapy. This embodiment of the invention can be carried out with a conventional device for administering a powder pharmaceutical composition by inhalation. For example, a powder mixture of the compound and a suitable powder base such as lactose or starch may be presented in unit dosage form in for example capsular or cartridges, e.g. gelatin, or blister packs, from which the powder may be administered with the aid of an inhaler.

Although the compositions of the invention may be directly ingested, inhaled, or otherwise administered, or used as an additive in conjunction with foods, it will be appreciated that they may be incorporated into a variety of foods and beverages. The terms “food,” “food product,” and “foodstuff” are used interchangeably herein and include, in addition to foods commonly consumed by humans and domesticated animals, functional foods, pharmafoods, designer foods, and nutraceuticals. Suitable foods and beverages include, but are not limited to, yogurts, ice creams, cheeses, baked products such as bread, biscuits and cakes, dairy and dairy substitute foods, soy-based food products, grain-based food products, starch-based food products, confectionery products, edible oil compositions, spreads, breakfast cereals, infant formulas, juices, power drinks, and the like. Within the scope of the term “foods” are to be included in particular food likely to be classified as functional foods, i.e. “foods that are similar in appearance to conventional foods and are intended to be consumed as part of a normal diet, but have been modified to physiological roles beyond the provision of simple nutrient requirements” (NFA Policy Discussion Paper 7/94).

As non-limiting examples, subject inactivated probiotic bacteria are in some embodiments incorporated into milk products, including liquid, solid, semi-solid, and powdered milk products. Thus, the invention provides a milk-based food product comprising subject inactivated probiotic bacteria. A subject food product includes milk, and any food products made from or containing milk, including, but not limited to, cheese, yogurt, butter, ice cream, and other frozen desserts, whipped toppings, cream, custard, pudding, nutritional drinks, infant formula, and milk chocolate. In some embodiments, the invention provides a food product comprising subject inactivated probiotic bacteria, where the food product is a milk product, and where the milk product is any of powdered infant formula; liquid infant formula; liquid milk; powdered milk; a flavored liquid milk; a flavored powdered milk; yogurt; a yogurt-based beverage; cheese; butter; cream; and the like; or combinations of the foregoing. A subject milk-based food product includes any food product that includes milk as a component, or that is made using milk.

Cheeses include any fresh or ripened cheese. Such cheese include, but are not limited to, Campesino, Chester, Danbo, Drabant, Herregard, Manchego, Provolone, Saint Paulin, Soft cheese, Taleggio, White cheese, Cheddar, Colby, Edam, Muenster, Gruyere, Emmenthal, Camembert, Parmesan, Romano, Mozzarella, Feta; cottage cheese; cream cheese, Neufchatel, etc.

In some embodiments, the milk-based food product is a processed cheese food product. Processed cheese food products include, but are not limited to, pizza, ready-to-eat dishes, toast, burgers, lasagna, dressing, sauces, cheese powder, cheese flavor, and processed cheese.

Subject inactivated probiotic bacteria are in some embodiments formulated with nutritional beverages, e.g., peptide-based preparations; beverages comprising nutrients that are easily absorbed by the gut epithelium, e.g., peptides, fatty acids, electrolytes, monosaccharides, disaccharides, and the like; nutritional beverages such as Ensure®; and the like.

In some embodiments, subject inactivated probiotic bacteria are incorporated into soy-based food products, including liquid, solid, semi-solid, and powdered soy food products. Thus, the invention provides a soy-based food product comprising subject inactivated probiotic bacteria. Soy-based food products include, but are not limited to, soy infant formula, soy “milk,” soy-based food bars, and the like. See, e.g., U.S. Patent Publication Nos. 20030219526 and 20030054087.

In some embodiments, subject inactivated probiotic bacteria are incorporated into grain-based food products, include food products that comprise whole grains, food products that comprise processed grains (e.g., milled grains, such as flour; parboiled grains; puffed grains; grains processed for breakfast cereals; and the like). Grain-based food products include those made using wheat, rice, oats, barley, rye, corn, amaranth, flax, millet, sorghum, triticale, or a combination of two or more grains. In some embodiments, subject inactivated probiotic bacteria are incorporated into flour-based food products, including breads, cookies, cakes, pastas, etc., made with milled grain(s). In some embodiments, subject inactivated probiotic bacteria are incorporated into gluten-free food products (e.g., food products free of wheat, rye and barley, or any of their derivatives), wheat-free food products, and casein-free food products.

In some embodiments, subject inactivated probiotic bacteria are incorporated into starch-based food products, e.g., products made using potato starch.

Additional Agents

Inactivated probiotic bacteria can be formulated with additional agents, which agents may be inert or active agents. For example, preservatives and other additives may also be present such as, for example, antimicrobial agents (e.g., antibacterials, antivirals, antifungals, etc.), antioxidants, chelating agents, and inert gases and the like. In some embodiments, inactivated probiotic bacteria are formulated with one or more additional therapeutic agent for the treatment of gastrointestinal inflammation, diarrhea, irritable bowel syndrome, microbial infection, allergy, etc.

Inactivated probiotic bacteria can be combined with conventional agents used for treatment of gastrointestinal inflammation, where appropriate. Exemplary agents used in conventional gastrointestinal inflammation therapy, such as those used in therapy for chronic gastrointestinal inflammation such as in IBD, include, but are not necessarily limited to, 5-aminosalicylate (5-ASA), sulfasalazine, corticosteroids, azathioprine, cyclosporine, and methotrexate, as well as tumor necrosis factor-α (TNF-α) antagonists, cytokines such as IL-10, or other drug useful in the treatment of chronic gastrointestinal inflammation.

Such additional agents can be administered separately or included in the inactivated probiotic bacteria composition. In addition, inactivated probiotic bacteria can be formulated with other agents, e.g., anti-inflammatory agents, with the proviso that such agents do not substantially interfere with the efficacy of inactivated probiotic bacteria. Exemplary agents include, but are not necessarily limited to, antacids, H2 blockers, proton pump inhibitors, and the like (e.g., famotidine; ranitidine hydrochloride, omeprazol, and the like). Suitable H2 blockers (histamine type 2 receptor antagonists) include, but are not limited to, Cimetidine (e.g., Tagamet, Peptol, Nu-cimet, apo-cimetidine, non-cimetidine); Ranitidine (e.g., Zantac, Nu-ranit, Novo-randine, and apo-ranitidine); and Famotidine (Pepcid, Apo-Famotidine, and Novo-Famotidine).

Subject inactivated probiotic bacteria can be formulated together with an immunosuppressive agent. Suitable immunosuppressive agents include, but are not limited to, a steroidal immunosuppressive agent, azathioprine, 6-mercaptopurine, methotrexate, cyclosporine, tacrolimus, mycophenolate mofetil, thalidomide, and the like.

Suitable TNF-α antagonists that can be formulated with a subject inactivated probiotic formulation include soluble TNF-α receptors, chimeric TNF-α receptors, antibodies to TNF-α, etc. Suitable TNF-α antagonists include, but are not limited to, ENBREL® (a dimeric fusion protein consisting of the extracellular ligand-binding portion of the human 75 kilodalton (p75) TNFR linked to the Fc portion of human IgG1; Smith et al. (1990) Science 248:1019-1023; Mohler et al. (1993) J. Immunol. 151:1548-1561; U.S. Pat. No. 5,395,760; and U.S. Pat. No. 5,605,690); Infliximab (REMICADE®; a chimeric monoclonal anti-TNF-α antibody that includes about 25% mouse amino acid sequence and about 75% human amino acid sequence; Elliott et al. (1993) Arthritis Rheum. 36:1681-1690; Elliott et al. (1994) Lancet 344:1105-1110; Baert et al. (1999) Gastroenterology 116:22-28); and Adalimumab (HUMIRA™; a human, full-length IgG1 monoclonal antibody that was identified using phage display technology. Piascik (2003) J. Am. Pharm. Assoc. 43:327-328); and the like.

Inactivated probiotic bacteria can be combined (e.g., formulated with) with conventional agents that treat diarrhea, e.g., loperamide (Imodium®, Imodium® A-D); bismuth subsalicylate; diphenyloxylate/atropine (Lomotil®); attapulgite (Kaopectate®); and the like.

Inactivated probiotic bacteria can be formulated with one or more antibiotics. Because the inactivated probiotic bacteria of the instant invention are non-viable or have reduced viability, an antibiotic can be included in the formulation without concern about adverse effects on probiotic viability. Antibiotics include, but are not limited to, Gentamicin; Vancomycin; Oxacillin; Tetracyclines; Nitroflurantoin; Chloramphenicol; Clindamycin; Trimethoprim-sulfamethoxasole; a member of the Cephlosporin antibiotic family (e.g., Cefaclor, Cefadroxil, Cefixime, Cefprozil, Ceftriaxone, Cefuroxime, Cephalexin, Loracarbef, and the like); a member of the Penicillin family of antibiotics (e.g., Ampicillin, Amoxicillin/Clavulanate, Bacampicillin, Cloxicillin, Penicillin VK, and the like); with a member of the Fluoroquinolone family of antibiotics (e.g., Ciprofloxacin, Grepafloxacin, Levofloxacin, Lomefloxacin, Norfloxacin, Ofloxacin, Sparfloxacin, Trovafloxacin, and the like); a member of the Macrolide antibiotic family (e.g., Azithromycin, Erythromycin, and the like); or Metronidazole; and the like.

Similarly, a therapeutically-effective concentration of an anti-fungal agent may be included in the inactivated probiotic formulation. Such anti-fungal agents include, but are not limited to: Clotrimazole, Fluconazole, Itraconazole, Ketoconazole, Miconazole, Nystatin, Terbinafine, Terconazole, and Tioconazole.

Inactivated probiotic bacteria can be formulated with one or more agents for treating allergy. Suitable therapeutic agents for the treatment of allergies which can be formulated with inactivated probiotic bacteria include, but are not limited to, antihistamines such as loratadine (Claritin®), fexofenadine (Allegra®), terfenadine; astemizole, cetirizine, hydroxyzine, diphenhydramine; leukotriene synthesis inhibitors zileutron (Zyflo®); leukotriene receptor antagonists such as zafirlukast (Accolate®), and montelukast; β-adrenergic agonists such as epinephrine, isoproterenol, isoetharine, metaproterenol, albuterol, terbutaline, bitolterol, pirbuterol, and salmeterol; proinflammatory cytokine antagonists; proinflammatory cytokine receptor antagonists; anti-CD23; anti-IgE; anticholinergics such as atropine and ipratropium bromide; immunomodulating drugs; glucocorticosteroids; steroid chemical derivatives; anti-cyclooxygenase agents; anti-cholinergic agents; methylxanthines, cromones; anti-CD4 reagents; anti-IL-5 reagents; anti-thromboxane reagents; anti-serotonin reagents; ketotiphen; cytoxin; cyclosporin; methotrexate; macrolide antibiotics; heparin; and low molecular weight heparin.

Nutraceutical Formulations

The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, food bars, soups, and beverages. The term “functional foods,” refers to foods that include “any modified food or food ingredients that may provide a health benefit beyond the traditional nutrients it contains.” Thus, by definition, pharmaceutical compositions comprising an inactivated probiotic bacterium include nutraceuticals. Also by definition, pharmaceutical compositions comprising inactivated probiotic bacteria include compositions comprising inactivated probiotic bacteria and a food-grade component. Inactivated probiotic bacteria may be added to food products to provide a health benefit.

Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; and the like. These foods are enhanced by the inclusion of an inactivated probiotic bacterium. For example, in the treatment of an inflammatory bowel disease, the normal diet of a patient may be supplemented by an inactivated probiotic bacterium nutraceutical formulation taken on a regular basis, e.g., at meal times, before meals, between meals, or after meals. As another example, in the treatment of diarrhea, inactivated probiotic bacteria are included in an electrolyte-containing beverage that the individual consumes periodically throughout the day when the individual is experiencing diarrhea.

The present invention provides compositions (e.g., nutraceutical compositions) comprising an inactivated probiotic bacterium and a food-grade pharmaceutically acceptable excipient. In many embodiments, subject nutraceutical compositions include one or more components found in food products. Thus, the instant invention provides a food composition and products comprising an inactivated probiotic bacterium and a food component. Suitable components include, but are not limited to, mono- and disaccharides; carbohydrates; proteins; amino acids; fatty acids; lipids; stabilizers; preservatives; flavoring agents; coloring agents; sweeteners; antioxidants, chelators, and carriers; texturants; nutrients; pH adjusters; emulsifiers; stabilizers; milk base solids; edible fibers; and the like. The food component can be isolated from a natural source, or can be synthesized. All components are food-grade components fit for human consumption.

Examples of suitable monosaccharides include sorbitol, mannitol, erythrose, threose, ribose, arabinose, xylose, ribulose, glucose, galactose, mannose, fructose, and sorbose. Non-limiting examples of suitable disaccharides include sucrose, maltose, lactitol, maltitol, maltulose, and lactose.

Suitable carbohydrates include oligosaccharides, polysaccharides, and/or carbohydrate derivatives. As used herein, the term “oligosaccharide” refers to a digestible linear molecule having from 3 to 9 monosaccharide units, wherein the units are covalently connected via glycosidic bonds. As used herein, the term “polysaccharide” refers to a digestible (i.e., capable of metabolism by the human body) macromolecule having greater than 9 monosaccharide units, wherein the units are covalently connected via glycosidic bonds. The polysaccharides may be linear chains or branched. Carbohydrate derivatives, such as a polyhydric alcohol (e.g., glycerol), may also be utilized as a complex carbohydrate herein. As used herein, the term “digestible” in the context of carbohydrates refers to carbohydrate that are capable of metabolism by enzymes produced by the human body. Examples of polysaccharides non-digestible carbohydrates are resistant starches (e.g., raw corn starches) and retrograded amyloses (e.g., high amylose corn starches). Non-limiting examples carbohydrates include raffinoses, stachyoses, maltotrioses, maltotetraoses, glycogens, amyloses, amylopectins, polydextroses, and maltodextrins.

Suitable fats include, but are not limited to, triglycerides, including short-chain (C₂-C₄) and long-chain triglycerides (C₁₆-C₂₂).

Suitable texturants (also referred to as soluble fibers) include, but are not limited to, pectin (high ester, low ester); carrageenan; alginate (e.g., alginic acid, sodium alginate, potassium alginate, calcium alginate); guar gum; locust bean gum; psyllium; xanthan gum; gum arabic; fructo-oligosaccharides; inulin; agar; and functional blends of two or more of the foregoing.

Suitable emulsifiers include, but are not limited to, propylene glycol monostearate (PGMS), sodium stearoyl lactylate (SSL), calcium stearoyl lactylate (CSL), monoglycerides, diglycerides, monodiglycerides, polyglycerol esters, lactic acid esters, polysorbate, sucrose esters, diacetyl tartaric acid esters of mono-diglycerides (DATEM), citric acid esters of monoglycerides (CITREM) and combinations thereof. Additional suitable emulsifiers include DIMODAN, including DIMODAN™ B 727 and DIMODAN™ PV, GRINDSTED™ CITREM, GRINDSTED™ GA, GRINDSTED™ PS such as GRINDSTED™ PS 100, GRINDSTED™ PS 200, GRINDSTED™ PS 300, GRINDSTED™ PS 400; RYLO™ (manufactured and distributed by DANISCO CULTOR), including RYLO™ AC, RYLO™ CI, RYLO™ LA, RYLO™ MD, RYLO™ MG, RYLO™ PG, RYLO™ PR, RYLO™ SL, RYLO™ SO, RYLO™ TG; and combinations thereof.

Edible fibers include polysaccharides, oligosaccharides, lignin and associated plant substances. Suitable edible fibers include, but are not limited to, sugar beet fiber, apple fiber, pea fiber, wheat fiber, oat fiber, barley fiber, rye fiber, rice fiber, potato fiber, tomato fiber, other plant non-starch polysaccharide fiber, and combinations thereof.

Suitable flavoring agents include natural and synthetic flavors, “brown flavorings” (e.g., coffee, tea); dairy flavorings; fruit flavors; vanilla flavoring; essences; extracts; oleoresins; juice and drink concentrates; flavor building blocks (e.g., delta lactones, ketones); and the like; and combinations of such flavors. Examples of botanic flavors include, for example, tea (e.g., preferably black and green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardamom, nutmeg, cassia bark, buchu, cinnamon, jasmine, haw, chrysanthemum, water chestnut, sugar cane, lychee, bamboo shoots, vanilla, coffee, and the like.

Suitable sweeteners include, but are not limited to, alitame; dextrose; fructose; lactilol; polydextrose; xylitol; xylose; aspartame, saccharine, cyclamates, acesulfame K, L-aspartyl-L-phenylalanine lower alkyl ester sweeteners, L-aspartyl-D-alanine amides; L-aspartyl-D-serine amides; L-aspartyl-hydroxymethyl alkane amide sweeteners; L-aspartyl-1-hydroxyethylalkane amide sweeteners; and the like.

Suitable anti-oxidants include, but are not limited to, tocopherols (natural, synthetic); ascorbyl palmitate; gallates; butylated hydroxyanisole (BHA); butylated hydroxytoluene (BHT); tert-butyl hydroquinone (TBHQ); and the like.

Suitable nutrients include vitamins and minerals, including, but not limited to, niacin, thiamin, folic acid, pantothenic acid, biotin, vitamin A, vitamin C, vitamin B₂, vitamin B 3, vitamin B₆, vitamin B₁₂, vitamin D, vitamin E, vitamin K, iron, zinc, copper, calcium, phosphorous, iodine, chromium, molybdenum, and fluoride.

Suitable coloring agents include, but are not limited to, FD&C dyes (e.g., yellow #5, blue #2, red #40), FD&C lakes; Riboflavin; β-carotene; natural coloring agents, including, for example, fruit, vegetable, and/or plant extracts such as grape, black currant, aronia, carrot, beetroot, red cabbage, and hibiscus.

Exemplary preservatives include sorbate, benzoate, and polyphosphate preservatives.

Suitable emulsifiers include, but are not limited to, diglycerides; monoglycerides; acetic acid esters of mono- and diglycerides; diacetyl tartaric acid esters of mono- and diglycerides; citric acid esters of mono- and diglycerides; lactic acid esters of mono- and diglycerides; fatty acids; polyglycerol esters of fatty acids; propylene glycol esters of fatty acids; sorbitan monostearates; sorbitan tristearates; sodium stearoyl lactylates; calcium stearoyl lactylates; and the like.

Suitable agents for pH adjustment include organic as well as inorganic edible acids. The acids can be present in their undissociated form or, alternatively, as their respective salts, for example, potassium or sodium hydrogen phosphate, potassium or sodium dihydrogen phosphate salts. Exemplary acids are edible organic acids which include citric acid, malic acid, fumaric acid, adipic acid, phosphoric acid, gluconic acid, tartaric acid, ascorbic acid, acetic acid, phosphoric acid and mixtures thereof.

Inactivated probiotic bacteria are present in the food product/nutraceutical formulation in an amount of from about 5% to about 90% by weight or by volume, e.g., from about 5% to about 7%, from about 7% to about 10%, from about 10% to about 15%, from about 15% to about 20%, from about 20% to about 25%, from about 25% to about 30%, from about 30% to about 40%, from about 40% to about 50%, from about 50% to about 60%, from about 60% to about 70%, from about 70% to about 80%, or from about 80% to about 90% by weight or by volume. In some embodiments, the inactivated probiotic bacteria present in the food product are homogenous, e.g., substantially all the inactivated probiotic bacteria in the food product are of the same species. In other embodiments, the inactivated probiotic bacteria in the food product comprise inactivated probiotic bacteria of two or more different species.

Where the food product is a beverage, the food product generally contains, by volume, more than about 50% water, e.g., from about 50% to about 60%, from about 60% to about 95% water, e.g., from about 60% to about 70%, from about 70% to about 80%, from about 80% to about 90%, or from about 90% to about 95% water.

Where the food product is a bar, the food product generally contains, by volume, less than about 15% water, e.g., from about 2% to about 5%, from about 5% to about 7%, from about 7% to about 10%, from about 10% to about 12%, or from about 12% to about 15% water.

In some embodiments, the food product is essentially dry, e.g., comprises less than about 5%, water.

Monosaccharides, disaccharides, and complex carbohydrates, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Soluble fibers, edible fibers, and emulsifiers, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each.

Other components discussed above, if present, are present in amounts ranging from about 0.001% to about 5% by weight of the composition.

Treatment Methods

The present invention provides methods of treating a variety of disorders, the methods generally involving administering to the individual suffering from the disorder a subject formulation. As used herein, the term “administration” includes self administration, e.g., ingestion. Disorders amenable to treatment by administration of a subject formulation include any disorder that is amenable to treatment with viable probiotic bacteria. Disorders amenable to treatment by administration of a subject formulation thus include, but are not limited to, gastrointestinal inflammation; microbial infections; diarrheal diseases; allergic disorders; antigen-stimulated inflammation; microbial infections; irritable bowel syndrome; non-alcoholic liver disease; and asthma.

The present invention provides methods of treating gastrointestinal inflammation. The methods generally involve administering to an individual in need thereof an effective amount of a subject formulation comprising inactivated probiotic bacteria. “Gastrointestinal inflammation” encompasses a variety of disorders, including, but not limited to, inflammatory bowel disease (IBD); irritable bowel syndrome; viral, bacterial, fungal, and parasitic colitis; colitis induced by environmental insults (e.g., gastrointestinal inflammation (e.g., colitis) caused by or associated with (e.g., as a side effect) a therapeutic regimen, such as administration of NSAIDS, chemotherapy, radiation therapy, and the like); colitis in conditions such as chronic granulomatous disease, celiac disease, celiac sprue; food allergies, e.g., lactose intolerance; gastritis; infectious gastritis or enterocolitis (e.g., Helicobacter pylori-infected chronic active gastritis) and other forms of gastrointestinal inflammation caused by an infectious agent, e.g., Cryptosporidium parvum infection, rotavirus gastroenteritis, tropical acute watery diarrhea, “traveler's diarrhea,” Clostridium difficile-induced colitis, Salmonella infections, Shigella infections.

A subject method of treating a gastrointestinal inflammatory disorder generally involves administering to an individual in need thereof a subject formulation in an amount effective to treat the disorder. The subject methods of treating a gastrointestinal inflammatory disorder include methods of treating individuals who have been diagnosed as having a gastrointestinal inflammatory disorder; methods of reducing the incidence of recurrence, or “flare up” of the disorder; methods of reducing the risk of flare up in an individual who has been diagnosed as having a gastrointestinal inflammatory disorder, has been treated for such by conventional therapies, and is in remission; and methods of treating a gastrointestinal inflammatory disorder in an individual who has failed to respond to conventional therapy for treating the disorder.

In a subject method for treating a gastrointestinal inflammatory disorder, an “effective amount” of a subject formulation is an amount that reduces the severity of a symptom and/or reduces a measurable parameter associated with the disease by at least about 10%, at least about 20%, at least about 25%, at least about 30%, at least about 40%, at least about 50%, at least about 60%, at least about 70%, at least about 80%, or at least about 90% or more, when compared with the symptom (e.g., the severity of the symptom), or when compared with the measurable parameter associated with the disease, in the absence of treatment with a subject formulation.

The present invention provides methods of treating allergic disorders. The term “allergic disorder” generally refers to a disease state or syndrome whereby the body produces an immune response to environmental antigens comprising immunoglobulin E (IgE) antibodies which evoke allergic symptoms such as itching, sneezing, coughing, respiratory congestion, rhinorrhea, skin eruptions and the like, as well as severe reactions, such as asthma attacks and systemic anaphylaxis. Examples of allergic diseases and disorders which can be treated by the methods of this invention include, but are not limited to, drug hypersensitivity, allergic rhinitis, bronchial asthma, ragweed pollen hayfever, anaphylactic syndrome, urticaria, angioedema, atopic dermatitis, erythema nodosum, erythema multiforme, Stevens-Johnson Syndrome, cutaneous necrotizing venulitis, bullous skin diseases, allergy to food substances and insect venom-induced allergic reactions, as well as any other allergic disease or disorder.

A subject method of treating an allergic disorder generally involves administering a subject formulation to an individual who is sensitized to an antigen (e.g., an allergen). A subject formulation is administered in an amount effective to treat the allergic disorder, e.g., to reduce production of IgE specific for the antigen (e.g., the allergen); to reduce the severity of a symptom of the allergic disorder; to reduce the amount of a conventional therapeutic agent that is required to treat the disorder; to reduce the frequency and/or severity of an allergic reaction to the allergen; and the like. Thus, e.g., an effective amount of a subject formulation is an amount that reduces the severity of a symptom and/or reduces a measurable parameter associated with the allergic disorder by at least about 10%, at least about 20%, at least about 25%, at least about 30%, at least about 40%, at least about 50%, at least about 60%, at least about 70%, at least about 80%, or at least about 90% or more, when compared with the symptom (e.g., the severity of the symptom), or when compared with the measurable parameter associated with the allergic disorder, in the absence of treatment with a subject formulation.

In some embodiments, an effective amount of a subject formulation reduces the level of serum IgE in an individual by at least about 10%, at least about 20%, at least about 25%, at least about 30%, at least about 40%, at least about 50%, at least about 60%, at least about 70%, at least about 80%, or at least about 90% or more, when compared with the level of serum IgE in the absence of treatment with a subject formulation. In some embodiments, an effective amount of a subject formulation reduces the severity of symptoms (e.g., reduces the frequency of coughing, sneezing, wheezing, etc.) by at least about 10%, at least about 20%, at least about 25%, at least about 30%, at least about 40%, at least about 50%, at least about 60%, at least about 70%, at least about 80%, or at least about 90% or more, when compared with the frequency of coughing, sneezing, wheezing, etc. in the absence of treatment with a subject formulation.

The present invention provides methods of treating a diarrheal disease. The methods generally involve administering to an individual in need thereof an effective amount of a subject formulation. Diarrheal diseases that are amenable to treatment with a subject method include diarrhea caused by a bacterial infection; diarrhea caused by a viral infection; diarrhea caused by a mixed bacterial and viral infection; radiation-induced diarrhea; and antibiotic-induced diarrhea. In the treatment of a diarrheal disease, an “effective amount” of a subject formulation is an amount that is effective to reduce the incidence and/or severity of a diarrheal disease, or that is effective to reduce the time to recover from the disease, by at least about 10%, at least about 20%, at least about 25%, at least about 30%, at least about 40%, at least about 50%, at least about 60%, at least about 70%, at least about 80%, or at least about 90% or more, when compared with the incidence, severity, or recovery time in the absence of treatment with a subject formulation.

In the treatment of a diarrheal disease, a subject formulation can be taken (administered) prophylactically. For example, a subject formulation can be taken immediately before, and/or during travel to a destination where the risk of contracting a diarrheal disease is high, thereby diminishing the risk that the individual will suffer from diarrhea. As another example, a subject formulation can be administered to an individual who is about to undergo radiation therapy for cancer, or who has recently undergone radiation therapy for cancer. For example, a subject formulation is administered to an individual from about 24 hours to about 72 hours before radiation treatment and/or from about 1 hour to about 24 hours following radiation treatment. Administration of a subject formulation can be initiated from about 1 hour to about 24 hours following radiation treatment, and continued for a period of time thereafter, e.g., for one day to about 2 weeks following radiation treatment. As another example, a subject formulation can be administered to an individual concurrently with a course of antibiotics, or immediately following a course of antibiotics, to reduce the incidence and/or severity of antibiotic-induced diarrhea.

The present invention provides a method of treating irritable bowel syndrome (IBS) in an individual. The methods generally involve administering to an individual in need thereof an effective amount of a subject formulation. In the treatment of IBS, an “effective amount” of a subject formulation is an amount that is effective to reduce the severity and/or incidence of one or more symptoms associated with IBS by at least about 10%, at least about 20%, at least about 25%, at least about 30%, at least about 40%, at least about 50%, at least about 60%, at least about 70%, at least about 80%, or at least about 90% or more, when compared with the incidence or severity in the absence of treatment with a subject formulation. Symptoms associated with IBS include bloating, gastrointestinal cramping, loose stool, frequent bowel movement, gas, and the like.

The present invention provides methods of treating non-alcoholic liver disease, including steatosis, non-alcoholic hepatitic steatohepatitis, and the like. The present invention further provides methods of reducing the risk that an individual will develop hepatic fibrosis or cirrhosis as a result of a non-alcoholic liver disease. The methods generally involve administering to an individual in need thereof an effective amount of a subject formulation. In some embodiments, an “effective amount” of a subject formulation is an amount that is effective to reduce the severity and/or incidence of one or more symptoms or parameters associated with non-alcoholic liver disease by at least about 10%, at least about 20%, at least about 25%, at least about 30%, at least about 40%, at least about 50%, at least about 60%, at least about 70%, at least about 80%, or at least about 90% or more, when compared with the incidence or severity of the symptom or the parameter in the absence of treatment with a subject formulation. In other embodiments, an “effective amount” of a subject formulation is an amount that is effective to liver function by at least about 10%, at least about 20%, at least about 25%, at least about 30%, at least about 40%, at least about 50%, at least about 60%, at least about 70%, at least about 80%, or at least about 90% or more, when compared with liver function in the absence of treatment with a subject formulation.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like. As one non-limiting example, levels of serum alanine aminotransferase (ALT) are measured, using standard assays. In general, an ALT level of less than about 45 international units is considered normal. In some embodiments, an effective amount of a subject formulation is an amount effective to reduce ALT levels to less than about 45 U/ml serum.

Whether a subject method is effective in reducing liver fibrosis can be determined by any of a number of well-established techniques for measuring liver fibrosis and liver function. Whether liver fibrosis is reduced is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of a subject treatment method can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

Secondary, or indirect, indices of liver function can also be used to evaluate the efficacy of treatment with the subject method. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

Dosages

Dosages that provide for a therapeutic effect range from about 1×10⁵ to about 1×10¹⁴, from about 5×10⁵ to about 5×10¹³, from about 1×10⁶ to about 1×10¹², from about 5×10⁶ to about 5×10¹¹, or from about 1×10⁷ to about 1×10¹⁰ bacteria per unit dosage form bacteria per dosing unit. A “dosing unit” or “unit dosage form,” which terms are used interchangeably herein, may be in the form of a tablet or capsule; a unit amount of a liquid or gel formulation; or, where the formulation is in the form of a food product or a nutraceutical, a serving size. In some embodiments, multiple doses of from about 1×10⁵ to about 1×10¹⁴, from about 5×10⁵ to about 5×10¹³, from about 1×10⁶ to about 1×10¹², from about 5×10⁶ to about 5×10¹¹, from about 1×10⁷ to about 1×10¹⁰, or from about 1×10⁸ to about 1×10⁹ bacteria are required to achieve a therapeutic effect. Thus, in some embodiments, a therapeutically effective dose is the amount of bacteria administered in two, three, four, five, six, seven, eight, nine, ten, or more dosing units.

For example, a therapeutically effective dose of bacteria is 1-5×10¹⁰ inactivated bacteria per packet, tablet, or capsule administered 1 to 4 times per day; 1-5×10¹¹ inactivated bacteria per packet, tablet, or capsule administered 1 to 4 times per day; 1-5×10¹² inactivated bacteria per packet, tablet, or capsule administered 1 to 4 times per day; 1×10¹³ inactivated bacteria per packet, tablet, or capsule administered 1 to 4 times per day; 1×10¹⁴ inactivated bacteria per packet, tablet, or capsule administered 1 to 4 times per day; 1-5×10¹⁰ inactivated bacteria per ml liquid formulation administered 1 to 4 times per day; 1-5×10¹¹ inactivated bacteria per ml liquid formulation; 1-5×10¹² administered 1 to 4 times per day; 1×10¹³ inactivated bacteria per ml liquid formulation administered 1 to 4 times per day; 1×10¹⁴ inactivated bacteria per per ml liquid formulation administered 1 to 4 times per day.

Routes of Administration

Conventional and pharmaceutically acceptable routes of administration for treatment of disorders such as allergy and gastrointestinal inflammation (e.g., chronic gastrointestinal inflammation such as that of IBD), include, but are not necessarily limited to, oral, intragastric, vaginal, rectal (e.g., enema, suppository), intranasal and other routes of effective inhalation routes, e.g., intrapulmonary. In general, gastrointestinal routes of administration are of particular interest in the present invention for treatment of gastrointestinal inflammation including, but not necessarily limited to oral, intranasal, intragastric, and rectal administration. Routes of administration of particular interest for the treatment of allergy include oral and inhalational routes of administration. Routes of administration of particular interest for the treatment of diarrheal diseases include oral and rectal routes of administration. Routes of administration for the treatment of microbial infection include oral, rectal, vaginal, and inhalational routes of administration. Routes of administration may be combined, if desired, or adjusted depending upon the inactivated probiotic bacteria and/or the desired therapeutic effect. The inactivated probiotic bacteria composition can be administered in a single dose or in multiple doses, and may encompass administration of additional doses, to elicit and/or maintain the desired effect.

Subject inactivated probiotic bacteria can be administered to a subject using any available conventional methods and routes suitable for delivery of conventional drugs. Methods and localized routes that further facilitate production of the anti-gastrointestinal inflammatory (e.g., anti-IBD) activity or allergy-reducing activity of the inactivated probiotic bacteria, e.g., at or near a site of inflammation or allergic reaction is of interest in the invention. In general, routes of administration contemplated by the invention include, but are not necessarily limited to, gastroenteral, enteral, vaginal, or inhalational. Gastroenteral routes of administration include, but are not necessarily limited to, oral and rectal (e.g., using an enema or a suppository) delivery. For the treatment of allergy, suitable routes of administration include inhalational routes (e.g., intranasal, oral).

Inhalational routes of administration (e.g., intranasal, oral, intrapulmonary, and the like) are particularly useful in some embodiments, e.g., in the treatment of allergy. Such means include inhalation of aerosol suspensions or insufflation of the polynucleotide compositions of the invention. Nebulizer devices, metered dose inhalers, and the like suitable for delivery of inactivated probiotic bacteria to the nasal mucosa, trachea and bronchioli are well-known in the art and will therefore not be described in detail here. For general review in regard to intranasal drug delivery, see, e.g., Chien, Novel Drug Delivery Systems, Ch. 5 (Marcel Dekker, 1992).

Timing of Administration

A subject formulation can be administered to a subject prior to onset of more severe symptoms (e.g., prior to onset of an acute inflammatory attack, prior to onset of an allergic reaction), or after onset of acute or chronic symptoms (e.g., after onset of an acute inflammatory attack, after onset of an allergic reaction). As such, inactivated probiotic bacteria can be administered at any time, and may be administered at any interval. Thus, in some embodiments, administration is episodic.

In other embodiments, administration is at regular intervals. In one embodiment, inactivated probiotic bacteria are administered about 5 minutes, about 15 minutes, about 30 minutes, about 1 hour, about 2 hours, about 4 hours, about 8 hours, about 12 hours, about 24 hours, about 2 days, about 4 days, about 8 days, about 16 days, about 30 days or 1 month, about 2 months, about 4 months, about 8 months, or about 1 year after initial onset of symptoms (e.g., gastrointestinal inflammation-associated symptoms) and/or after diagnosis of a disorder (e.g., gastrointestinal inflammation, irritable bowel syndrome, etc.) in the subject, or after initial onset of an allergic reaction. As described in more detail below, the invention also provides for administration of subsequent doses of inactivated probiotic bacteria.

When multiple doses are administered, subsequent doses are administered within about 16 weeks, about 12 weeks, about 8 weeks, about 6 weeks, about 4 weeks, about 2 weeks, about 1 week, about 5 days, about 72 hours, about 48 hours, about 24 hours, about 12 hours, about 8 hours, about 4 hours, or about 2 hours or less of the previous dose. In one embodiment, inactivated probiotic bacteria are administered at intervals ranging from at least every two weeks to every four weeks (e.g., monthly intervals) in order to maintain the maximal desired therapeutic effect (e.g., to provide for maintenance of relief from symptoms). In another embodiment, inactivated probiotic bacteria are administered at intervals ranging from once per week, to twice per week, to three times per week, to once per day, to twice per day, or to three times per day.

In view of the teaching provided by this disclosure, those of ordinary skill in the clinical arts will be familiar with, or can readily ascertain, suitable parameters for administration of inactivated probiotic bacteria according to the invention.

Determining Therapeutic Efficacy

Where the disorder is gastrointestinal inflammation, the effectiveness of therapy can be monitored by monitoring the reduction of disease activity in the subject. Reduction in disease activity can be monitored by, for example, monitoring reduction of incidence of diarrhea or volume of stool, reduction of rectal bleeding, reduction of weight loss, reduction of size or number of colon lesions, reduction or opening of strictures, reduction or closure of fistulae, and the like. Therapeutic effectiveness can also be measured by for example, a decrease in C-reactive protein (CRP) level, a decrease in anti-neutrophil cytoplasmic antibodies (ANCA) in a biological sample, a decrease in erythrocyte sedimentation rate (ESR), a decrease in colonic myeloperoxidase (MPO) activity, reduction of anemia (as detected by, for example, hemoglobin levels, and the like), or other conventional indicator of gastrointestinal inflammation. Many of these methods for assessing therapeutic efficacy can be accomplished through endoscopy or through blood tests. Methods for monitoring gastrointestinal inflammation are well known in the art and well within the skill and knowledge of the ordinarily skilled artisan. Indicators of efficacy of the treatment can include a reduction in severity and/or absence of symptoms, an increase in the number of symptom-free days per time period (e.g., per week, per month) and/or a reduction in the need for conventional medications.

Where the disorder is allergy, the efficacy of the treatment can be monitored according to clinical protocols well known in the art for monitoring the treatment of allergic disorders. For example, such clinical parameters as allergy symptoms (itching, sneezing, coughing, respiratory congestion, rhinorrhea, skin eruption, etc.), assays and skin prick tests (wheal and flare response) to known allergens and serum levels of IgE and allergy-associated cytokines (e.g., interleukin-4, interleukin-5) can be monitored for determining efficacy. Indicators of efficacy of the treatment can include a reduction in severity and/or absence of symptoms, an increase in the number of symptom-free days per time period (e.g., per month) and/or a reduction in the need for conventional medications such as decongestants, anti-histamines, mast cell stabilizers and corticosteroids.

If the treatment of this invention is carried out in conjunction with immunotherapy, efficacy can be evaluated by observing an increase in tolerated dose of a given allergen(s). These parameters can be monitored weekly or monthly, as well as at greater time intervals (e.g., every 3-6 months). In a particular example, clinical parameters that can be monitored for asthma can include the number and severity of attacks as determined by symptoms of wheezing, shortness of breath and coughing. The measurement of airway resistance by the use of respiratory spirometry, the extent of disability and the dependence on immunosuppressive medications or bronchodilators can also be determined.

The efficacy of treatment for preventing an allergic disorder in a subject not known to have an allergic disorder, but known to be at risk of developing an allergic disorder, can be determined by evaluating clinical parameters such as allergy symptoms (itching, sneezing, coughing, respiratory congestion, rhinorrhea, skin eruption, etc.), assays and skin prick tests (wheal and flare response) to known allergens and serum levels of IgE and allergy-associated cytokines (e.g., interleukin-4, interleukin-5), over time following administration of the nucleic acid or fusion protein of this invention. This time interval can be very short (i.e, minutes/hours) or very long (i.e., years/decades). The determination of who would be at risk for the development of an allergic disorder would be made based on current knowledge of the known risk factors for a particular allergic disorder as would be familiar to clinicians and researchers in this field, such as a particularly strong family history of an allergic disorder or exposure to or acquisition of factors or conditions (i.e., environmental factors or conditions) which are likely to lead to development of an allergic disorder.

Where the disorder is diarrhea, the efficacy of a particular treatment is determined by monitoring symptoms reported by the individual or observed by a clinician. Efficacy can be assessed by determining the number of bacteria and/or virus in the stool of an individual who has diarrhea.

Reduction of Risk of Subsequent Disease

The methods of the invention can also provide for reduced risk of other conditions for which gastrointestinal inflammation is a risk factor. For example, ulcerative colitis is a risk factor for colonic carcinoma. Thus, treatment of ulcerative colitis (e.g., by reduction of inflammation) according to the methods of the invention also reduces the risk of colonic cancer (e.g., colonic carcinoma, colonic adenoma, and the like). The methods of the invention can thus be applied as prophylactic measure to prevent or reduce the risk of onset of colonic carcinoma, particularly in those patients that are high risk of colon cancer.

Established risk factors for colon cancer in those patients having ulcerative colitis include long duration of the disease, large extent of the disease, low activity of the disease, young age at onset, presence of complicating primary sclerosing cholangitis or stenotic disease and possibly lack of adequate surveillance, inadequate pharmacological therapy, folate deficiency and smoking. Crohn disease is associated with an increased risk of colorectal carcinoma in patients with long-standing disease, strictures and fistulae under the condition that the colon is involved, tumors of the small intestine may occur occasionally. Thus treating using inactivated probiotic bacteria according to the invention can be of particular benefit in these patients.

Combination Therapy

Inactivated probiotic bacteria can be administered in combination therapy with additional therapeutic agents. For example, in some embodiments, the methods provide for treatment of a gastrointestinal inflammatory disorder, a diarrheal disease, a microbial infection, an allergic disorder, etc., involving administering inactivated probiotic bacteria, and a second therapeutic agent.

Inactivated probiotic bacteria can be administered in combination therapy with conventional agents used for treatment of gastrointestinal inflammation, where appropriate. Exemplary agents used in conventional gastrointestinal inflammation therapy, such as those used in therapy for chronic gastrointestinal inflammation such as in IBD, include, but are not necessarily limited to, 5-aminosalicylate (5-ASA), sulfasalazine, corticosteroids, azathioprine, cyclosporine, and methotrexate, as well as tumor necrosis factor-α (TNF-α) antagonists (including antibodies specific for TNF-α; soluble TNF receptor; and the like), cytokines such as IL-10, or other drug useful in the treatment of chronic gastrointestinal inflammation. Such additional agents can be administered separately or included in the inactivated probiotic bacteria formulation. In addition inactivated probiotic bacteria can be administered in combination therapy with other anti-inflammatory agents, with the proviso that such agents do not substantially interfere with the efficacy of inactivated probiotic bacteria. Exemplary agents include, but are not necessarily limited to, antacids, H2 blockers, proton pump inhibitors, and the like (e.g., famotidine, ranitidine hydrochloride, omeprazole, and the like).

Suitable H2 blockers (histamine type 2 receptor antagonists) include, but are not limited to, Cimetidine (e.g., Tagamet, Peptol, Nu-cimet, apo-cimetidine, non-cimetidine); Ranitidine (e.g., Zantac, Nu-ranit, Novo-randine, and apo-ranitidine); and Famotidine (Pepcid, Apo-Famotidine, and Novo-Famotidine).

Subject inactivated probiotic bacteria can be administered in combination therapy with an immunosuppressive agent. Suitable immunosuppressive agents include, but are not limited to, a steroidal immunosuppressive agent, azathioprine, 6-mercaptopurine, methotrexate, cyclosporine, tacrolimus, mycophenolate mofetil, thalidomide, and the like.

Suitable TNF-α antagonists that can be administered in combination therapy with a subject inactivated probiotic formulation include soluble TNF-α receptors, chimeric TNF-α receptors, antibodies to TNF-α, etc. Suitable TNF-α antagonists include, but are not limited to, ENBREL® (a dimeric fusion protein consisting of the extracellular ligand-binding portion of the human 75 kilodalton (p75) TNFR linked to the Fc portion of human IgG1; Smith et al. (1990) Science 248:1019-1023; Mohler et al. (1993) J. Immunol. 151:1548-1561; U.S. Pat. No. 5,395,760; and U.S. Pat. No. 5,605,690); Infliximab (REMICADE®; a chimeric monoclonal anti-TNF-α antibody that includes about 25% mouse amino acid sequence and about 75% human amino acid sequence; Elliott et al. (1993) Arthritis Rheum. 36:1681-1690; Elliott et al. (1994) Lancet 344:1105-1110; Baert et al. (1999) Gastroenterology 116:22-28); and Adalimumab (HUMIRA™; a human, full-length IgG1 monoclonal antibody that was identified using phage display technology. Piascik (2003) J. Am. Pharm. Assoc. 43:327-328); and the like.

Subject inactivated probiotic bacteria are in some embodiments administered in combination therapy with a nutritional beverage, e.g., peptide-based liquid preparations; beverages comprising nutrients that are easily absorbed by the gut epithelium, e.g., peptides, fatty acids, electrolytes, monosaccharides, disaccharides, and the like; nutritional beverages such as Ensure®, Sustacal®, etc.; and the like.

Inactivated probiotic bacteria can be administered in combination therapy with conventional agents that treat diarrhea, e.g., loperamide (Imodium®, Imodium® A-D); bismuth subsalicylate; diphenyloxylate/atropine (Lomotil®); attapulgite (Kaopectate®); and the like.

Inactivated probiotic bacteria can be administered in combination therapy with one or more antibiotics, e.g., for the treatment of Cryptosporidium parvum infection, Shigella infection, or Salmonella infections. Antibiotics include, but are not limited to, Gentamicin; Vancomycin; Oxacillin; Tetracyclines; Nitroflurantoin; Chloramphenicol; Clindamycin; Trimethoprim-sulfamethoxasole; a member of the Cephlosporin antibiotic family (e.g., Cefaclor, Cefadroxil, Cefixime, Cefprozil, Ceftriaxone, Cefuroxime, Cephalexin, Loracarbef, and the like); a member of the Penicillin family of antibiotics (e.g., Ampicillin, Amoxicillin/Clavulanate, Bacampicillin, Cloxicillin, Penicillin VK, and the like); with a member of the Fluoroquinolone family of antibiotics (e.g., Ciprofloxacin, Grepafloxacin, Levofloxacin, Lomefloxacin, Norfloxacin, Ofloxacin, Sparfloxacin, Trovafloxacin, and the like); a member of the Macrolide antibiotic family (e.g., Azithromycin, Erythromycin, and the like); or metronidazol.

Similarly, a therapeutically-effective concentration of an anti-fungal agent may be administered in combination therapy with a subject-inactivated probiotic formulation. Such anti-fungal agents include, but are not limited to: Clotrimazole, Fluconazole, Itraconazole, Ketoconazole, Miconazole, Nystatin, Terbinafine, Terconazole, and Tioconazole.

Inactivated probiotic bacteria can be administered in combination therapy with a second therapeutic agent for the treatment of allergy. Therapeutic agents for the treatment of allergy include, but are not limited to, a steroid, an anti-histamine, an anti-inflammatory agent, a leukotriene synthesis inhibitor, an immunosuppressant, a bronchodilator, a vasoconstrictor, a decongestant, a leukotriene inhibitor, and the like.

Suitable therapeutic agents for the treatment of allergies which can be used in combination therapies with an agent of the instant invention include, but are not limited to, antihistamines such as loratadine (Claritin®), fexofenadine (Allegra®), terfenadine; astemizole, cetirizine, hydroxyzine, diphenhydramine; leukotriene synthesis inhibitors zileutron (Zyflo®); leukotriene receptor antagonists such as zafirlukast (Accolate®), and montelukast; β-adrenergic agonists such as epinephrine, isoproterenol, isoetharine, metaproterenol, albuterol, terbutaline, bitolterol, pirbuterol, and salmeterol; proinflammatory cytokine antagonists; proinflammatory cytokine receptor antagonists; anti-CD23; anti-IgE; anticholinergics such as atropine and ipratropium bromide; immunomodulating drugs; glucocorticosteroids; steroid chemical derivatives; anti-cyclooxygenase agents; anti-cholinergic agents; methylxanthines, cromones; anti-CD4 reagents; anti-IL-5 reagents; anti-thromboxane reagents; anti-serotonin reagents; ketotiphen; cytoxin; cyclosporin; methotrexate; macrolide antibiotics; heparin; and low molecular weight heparin.

Inactivated probiotic bacteria and an additional therapeutic agent may be administered in the same formulation or in separate formulations. Where the inactivated probiotic bacteria and the additional therapeutic agents are administered in separate formulations, they may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

Subjects Suitable for Treatment

Subjects suitable for treatment with the formulations and methods of the instant invention include any individual who has been diagnosed as having a gastrointestinal inflammatory disorder. Also suitable are individuals who failed treatment with one or more standard therapies for treating a gastrointestinal inflammatory disorder. Also suitable are individuals who have been treated for a gastrointestinal inflammatory disorder, and are in remission. Suitable individuals include immunocompetent as well as immunocompromised individuals.

Subjects suitable for treatment with the formulations and methods of the instant invention include any individual who has been diagnosed as having an allergy. Subjects amenable to treatment using the methods and agents described herein include individuals who are known to have allergic hypersensitivity to one or more allergens. Subjects amenable to treatment include those who have any of the above-mentioned allergic disorders. Also amenable to treatment are subjects that are at risk of having an allergic reaction to one or more allergens. Also suitable are individuals who failed treatment with one or more standard therapies for treating an allergic disorder.

Subjects suitable for treatment with a subject formulation and method include individuals suffering from IBS.

Subjects suitable for treatment with a subject formulation and method include individuals having a microbial infection. In some embodiments, the individual is immunocompromised. Immunocompromised individuals include CD4⁺ T cell deficient individuals; individuals who are immunocompromised following a course of cancer chemotherapy; individuals having an inherited immunodeficiency; individuals who are immunocompromised following a course of radiation therapy; and the like.

In some embodiments, an immunocompromised individual is a CD4⁺-deficient individuals, e.g., individuals who have lower than normal numbers of functional CD4⁺ T lymphocytes. As used herein, the term “immunocompetent” refers to an individual having CD4⁺ T lymphocyte levels and function(s) within the normal range in the population, for humans, typically 600 to 1500 CD4⁺ T lymphocytes per mm³ blood. CD4⁺-deficient individuals who have an acquired immunodeficiency, or a primary immunodeficiency. An acquired immunodeficiency may be a temporary CD4⁺ deficiency, such as one caused by radiation therapy, or chemotherapy. In some embodiments, an immunocompromised individual suitable for treatment has a bacterial infection, a viral infection, or a helminth infection (e.g., a Cryptosporidium parvum infection).

Subjects suitable for treatment with a subject formulation and method include individuals having diarrhea. Such individuals include those infected with a virus, bacteria, or combination of virus and bacteria, who have diarrhea as a result of the infection; individuals who are being treated with antibiotics and who have diarrhea as a result; individuals who have been treated for cancer with radiation and who have diarrhea as a result. Subjects suitable for treatment with a subject formulation and method include individuals at risk of developing diarrhea. Individuals at risk of developing diarrhea include individuals traveling in an area where drinking water that is contaminated with viruses and/or bacteria that cause diarrhea is prevalent; individuals who are about to be treated with a course of antibiotics or who are undergoing treatment with a course of antibiotics; and individuals who are undergoing radiation therapy for cancer.

Subjects suitable for treatment with a subject formulation and method include individuals who have been diagnosed with non-alcoholic liver disease. Such subjects include individuals in whom non-alcoholic liver disease has given rise to fibrosis or cirrhosis.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s, second(s); min, minute(s); hr, hour(s); i.g., intragastric; i.r., intrarectal/intrarectally; cfu, colony forming units; and the like.

### Example 1

### Treatment of DSS-Induced Colitis

Materials and Methods

Animals

Balb/c, C57B1/6 (B6), 129XB6F₂, congenic mice bearing the C3H-Tlr4^{Lps-d} mutation (i.e., resistant to LPS) on the Balb/c background, and IL-10 deficient mice were purchased from The Jackson Laboratory (Bar Harbor, Me.). MyD88 (B6), TLR2 (B6) and TLR9 (129xB6F₂) deficient mice were used as described and are currently bred in the UCSD vivarium. Takeda et al. (2003) Annu Rev Immunol 21:335-376; and Hemmi et al. (2000) Nature 408:740-745.

Probiotic Preparations

Probiotic bacteria (VSL-3) were purchased from VSL Pharmaceutical Inc. (Gaithersburg, Mass.). Each packet contains viable lyophilized gram⁺ bacteria of four strains of lactobacilli (L. casei, L. plantarum, L. acidophilus, and L. delbrueckii subsp bulgaricus), three strains of bifidobacteria (B. longum, B. breve, and B. infantis), and one strain of Streptococcus salivarius subsp. Thermophilus. Original packets (450×10⁹ CFU per packet) were irradiated with 1.2 M rad using a ¹³⁷Cs source at a rate of 8 Gy/min overnight. Heat-killed VSL were prepared by resuspending viable probiotics in PBS at 28×10⁸ CFU/ml followed by incubation for 30 min at 100° C. (heat block), centrifuged at 8,000 RPM for 5 min, washed in PBS and resuspended in fresh PBS prior to their administration. All VSL preparations were resuspended in phosphate-buffered saline (PBS) at a final concentration of 28×10⁸ CFU/ml and then cultured as described. Madsen et al. (2001) Gastroenterology 121:580-591. The resulting viability was determined by plating the cells on MRS-agar plates (Difco Laboratories, Detroit, Mich.) under anaerobic conditions for 16 hours at 37° C. No colonies were detected in the irradiated or heat-killed VSL while 22.1×10⁸±6.1 CFU/ml were recovered for viable (untreated) VSL (28×10⁸ as specified by the manufacturer).

Genomic DNA and Oligodeoxynucleotide Preparations

Genomic DNA was isolated from VSL-3 packets (VSL Pharmaceutical) and from E. coli (DH5α, Invitrogen, Carlsbad, Calif.) using DNA Isolation Kit (Qiagen, Valencia, Calif.) according to the manufacturer's instructions. The purity of DNA was confirmed by measuring the UV 260/280-absorbance ratio (<1.8). LPS levels in the DNA preparations were detected by limulus amebocytes lystate (BioWhittaker Inc., Wakersville Md.) and were <0.2 EU per μg of DNA.

Cytosine methylation of CpG dinucleotides in isolated probiotic DNA was performed by Sss I methylase (CpG methylase) (New England BioLabs, Beverly, Mass.) according to the manufacturer's instructions. Methylated DNA was extracted with phenol/chloroform for deproteination. Methylation of DNA was confirmed by digestion with restriction endonuclease BstUl followed by agarose gel electrophoresis.

Calf thymus DNA was purchased from Sigma (St Louis Mo.). Immunostimulatory oligodeoxynucleotide (ISS-ODN) (5′-TGACTGTGAACGTTCGAGATGA-3′; SEQ ID NO:01) and the control ODN (5′-TGACTGTG AAGGTTAGAGATGA-3; SEQ ID NO:02) on a phosphothioate backbone and were purchased from Tri-Link (San Diego, Calif.). Rachmilewitz et al. (2002) Gastroenterology 122:1428-1441.

To generate DNA-free probiotics, bacteria (VSL-3) were suspended in saline and disrupted by sonication. Bacterial lysates were incubated with DNase I (Roche, Indianapolis, Ind.) (10 U/ml) in the presence of 1 mM MgCl₂ on ice for 2 hrs. Elimination of DNA was confirmed by ethidium bromide staining on a 1% TAE agarose-gel.

Colitis Models

To induce dextran sodium sulfate (DSS) colitis, DSS (Sigma) was given in the drinking water for 7 days. Preliminary studies were performed to identify the concentration of DSS in the drinking water required to elicit a similar disease activity score in different mouse strains. 3.5% of DSS in Balb/c mice was equivalent to 1.5% DSS in B6 mice and to 1.75% DSS in 129/B6 mice.

Trinitrobenzenesulfonic acid (TNBS) colitis was induced in 8 week old, Balb/c mice by rectal instillation of 0.5 mg/mouse of 2,4,6-trinitrobenzene sulfonic acid (Sigma) dissolved in 0.1 ml of 50% ethanol as described. Rachmilewitz et al. (2002), supra.

Mice were sacrificed 7 days after the induction of colitis. All studies were performed in a blind fashion.

Probiotics and Various DNAs Treatment Protocols

Probiotics, including live probiotics, irradiated probiotics, and heat killed probiotics, were intragastrically (i.g.) given starting 10 days prior to the induction of colitis and for 7 days thereafter. In preliminary studies, mice were treated daily by i.g. administration of 0.28×10⁸, 2.8×10⁸ or 28×10⁸ CFU of irradiated probiotics per mouse per day. The administration of 2.8×10⁸ CFU/mouse/day was sufficient to inhibit colitis in Balb/c mice, whereas the administration of 28×10⁸ CFU/mouse/day was required to inhibit colitis in the other mouse strains. In some experiments chloroquine (10 mg/kg) (Sigma) was injected s.c. daily after the i.g. administration of viable or irradiated probiotics (2.8×10⁸ CFU/mouse/day).

Various DNA preparations (ISS-ODN and control-ODN, 30 μg/mouse; probiotic DNA, methylated probiotic DNA, E. coli DNA and calf thymus DNA, 50 μg/mouse) and DNase treated probiotics (i.e., the amount of microorganisms that yielded 50 μg of probiotic DNA) were s.c. injected 2 hrs prior to the administration of DSS or TNBS. In another experiment 50 μg of these DNA preparations were administered i.g. or intrarectally (i.r.) 2 hrs prior to DSS administration. In the IL-10 deficient colitis model, ten-week old mice were treated s.c. once a week with the various DNA preparations (see above), and this treatment continued for 4 weeks as described (Rachmilewitz et al. (2002) supra). The disease-activity score, histological score, and colonic myeloperoxidase (MPO) activity were determined as described (Rachmilewitz et al. (2002) supra).

Effect of Probiotics on Chronic DSS Induced Colitis:

To evaluate whether probiotics are effective not only in the prevention but also in the treatment of colitis the following experiment was performed: Mice were treated for 7 days with DSS 3.5% added to the drinking water. From the 8^{th} day until sacrifice on day 15, the concentration of DSS in the drinking water was reduced to 1.75%. During the 15 days of the experiment, 2 groups of mice were treated daily i.g. with viable or with irradiated probiotics 2.8×10⁸ CFU. A third group was treated s.c. on day 8 with ISS-ODN (10 μg) and a fourth control group was treated i.g. daily with 0.2 ml of saline. Mice were observed for rectal bleeding, weighed and sacrificed on day 15. The colon was isolated, weighed, sections were taken for histology and mucosal samples were obtained for MPO determination.

Effect of Chloroquine on Normal Flora and on Probiotic Bacterial Strains

To test whether chloroquine has antimicrobial activity on probiotics and on the commensal flora, mice were treated s.c. daily for 7 days with 10 mg/kg of chloroquine (Sigma). Control group was treated s.c. daily with 0.2 ml of saline. After 7 days, stool samples were collected, homogenized and cultured on blood, Maconkey, phenylethanol, chocolate, M.R.S., and anaerobic agars. In another experiment, all strains of fecal flora and all probiotic strains were tested for susceptibility to chloroquine by the agar dilution method. Concentrations tested ranged from 0.3-250 μg/ml of choloroquine. No significant differences were observed as to the identity or quantity of bacterial strains grown from the stool of chloroquine and saline treated mice. The flora that grew included: Bacillus spp.; Enterococcus sp.; Escherichia coli; Diphtheroid sp.; Lactobacillus sp.; and Bacteroides sp. All strains, inclusive of the probiotic strains, grew on all plates including those containing 250 μg/ml. The MIC of all bacterial strains is therefore >250 μg/ml.

Activation of Bone Marrow Derived Macrophages (BMDM) by DNAs

Bone marrow derived macrophages were prepared from Balb/c mice as described (Rachmilewitz et al. (2002) supra). BMDM (1×10⁶) were incubated for 48 hrs with 0.1-10 μg/ml of the various DNA preparations. The levels of IL-6 and IL-12 in the supernatants were determined by enzyme linked immunosorbent assay (ELISA; BD-Pharmingen, San Diego, Calif.) 24 hours post-stimulation.

Detection of Absorbed DNAs in Mice

For the detection of plasmid DNA (pDNA), one mg of pBudCE4 (Invitrogen) was administered i.g. or i.r. to Balb/c mice. Mice were sacrificed at various time points after pDNA administration and DNA was extracted from liver and spleen using DNeasy Tissue Kit (Qiagen). For the detection of probiotic DNA, 28×10⁸ CFU of irradiated probiotics was delivered i.g. for 10 days before DSS administration and for 7 days thereafter as described above. Ten μg each of isolated DNA was run on 1% TAE-agarose gel, transferred onto Hybond-N⁺ membrane (Amersham, Piscataway, N.J.), and hybridized to ³²P-labeled pDNA or VSL DNA using hybridization solution (Clontech, Palo Alto, Calif.). The hybridized membrane was exposed to X-ray film (Kodak, Rochester, N.Y.) at −80° C. overnight.

Signaling Assays

Translocation of nuclear factor-B (NF-κB) was detected by EMSA as described (Lee et al. (2000) J Leukoc Biol 68:909-915). For JNK and IKK kinase assays, lysates of cells or tissues were prepared, and JNK1 or IKK were immunoprecipitated using anti-JNK1 or anti-IKK antibodies (Santa Cruz Biotech, Santa Cruz, Calif.). The kinase activities were determined by an in vitro kinase assay using GST-cJun for JNK or GST-IκBα for IKK as a substrate, respectively (Lee et al. (2000) supra).

Statistical Analysis

Data are expressed as ±SEM. Statistical analyses for significant differences were performed according to parametric, Student t test (MPO activity), non-parametric, Mann-Whitney test (disease activity score and histological score). In some assays Chi square test was applied.

Viability Assays

The viability of the bacteria was determined by plating on MRS-agar plates (MRS: DeMan, Rogosa, Sharpe; Difco laboratories). The VSL suspensions of various treatments were serially diluted (1:10) and a 200 μl aliquot of each dilution was plated on MRS-agar plates. The plates were incubated anaerobically for 16 hours at 37° C. The numbers of colonies on the plates were counted and multiplied by the dilution factor. No colonies were detected in the suspension of γ-irradiated or heat-treated bacteria, while 21.1×10⁸±7.1 cfu/ml was recovered from non-treated VSL (28×10⁸ as specified by the manufacturer).

DSS Induced Colitis

A mouse model of colitis was used and assessed, as described. Rachmilewitz et al. (2002) Gastroenterology 122:1428-1441. Colitis was induced by adding dextran sodium sulfate (DSS, Sigma), 3.5% to the drinking water, and allowing them to drink ad libitum. Seven days after induction of colitis, mice were weighed and inspected for diarrhea and rectal bleeding. The mice were sacrificed, and the entire colon was dissected and its length measured and weighed. Scores were again defined as follows: Changes in body weight: No loss—0; 5 to 10%—1; 10 to 25%, —2; 15 to 20%, —3; >20%—4. Hemoccult: No blood, —0; positive, —2; gross blood, —4. Mucosal samples were processed for determination of MPO activity according to: Bradley (1982) J Invest Dermatol 78:206-9.

Histological Score

When indicated, sections from the distal colon were fixed in buffered formalin and routine 5 μm sections were prepared and stained with hematoxylin and eosin. Stained sections were examined blindly and scored. The scoring system took into account the depth of the ulcer, the extent of the ulcer, presence of inflammation, extent of inflammation, and location of fibrosis. Minimal score was 0 and maximal score was 20. Scoring was as follows for depth of the ulcer: 0=no ulcer; 1—mucosal involvement; 2—mucosal+submucosal involvement; 3=penetration of muscularis propria; 4=full thickness involvement. Scoring was as follows for extent of the ulcer: 0=no ulcer; 1=punctate; 2=minimal; 3=moderate; 4=widespread. Scoring was as follows for presence of inflammation: 0=none; 1=minimal; 2=mild; 3=moderate; 4=severe. Scoring was as follows for extent of inflammation: 0=none; 1=mucosal; 2=mucosal+submucosal involvement; 3=mucosal+submucosal+muscle penetration; 4=full thickness involvement. Scoring was as follows for location of fibrosis: 0=none; 1=mucosa only; 2=mucosa+submucosa; 3=including muscle layer; 4=full thickness fibrosis.

Determination of MPO Activity

Fifty mg colonic mucosal scrapings were homogenized with a polytron (Kinematica GmbH, Krienz-Luzem, Switzerland) in ice-cold hexadecyltrimethyl ammonium bromide (0.5%) in 50 mM phosphate buffer, pH 6.0. The homogenate was sonicated for 10 seconds, freeze-thawed three times, and centrifuged for 15 minutes. An aliquot of the supernatant was taken for determination of myeloperoxidase (MPO) enzyme activity, as described (Rachmilewitz et al. (2002) supra).

Results

Probiotic and E. coli DNA Have Immunostimulatory Activities

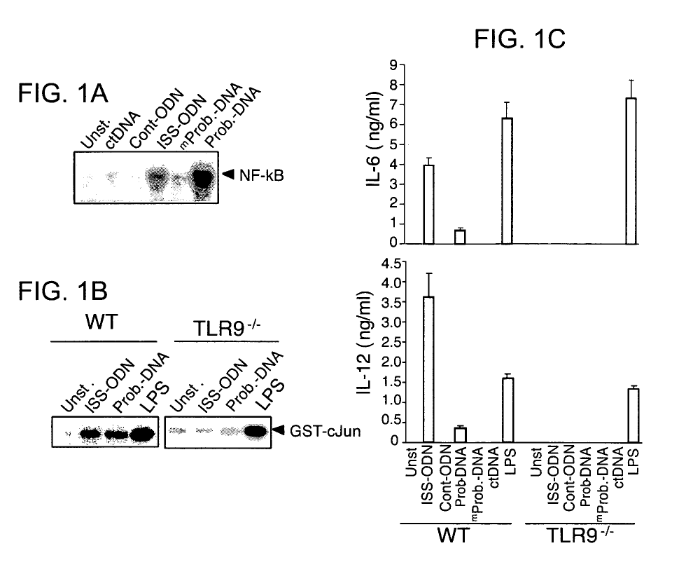

In order to evaluate the immunostimulatory properties of probiotic DNA, we assessed the ability of probiotic DNA to activate NF-kB and JNK, two major signaling pathways involved in TLR activation. Probiotic DNA, but not methylated probiotic DNA or calf thymus DNA, activated NF-kB (EMSA), as did ISS-ODN but not control-ODN (FIG. 1A). Similar results were obtained for JNK activation (FIG. 1B). The activation of these signaling pathways resulted in the induction of IL-12 (p40) and IL-6, which was mediated via TLR9 as both probiotic DNA and ISS-ODN did not induce the secretion of p40 or IL-6 in TLR9 null macrophages (FIG. 1C). Similar immunostimulatory profile was observed with E. coli genomic DNA.

FIGS. 1A-C: Probiotic DNA has immunostimulatory activities that depend on TLR9. BMDM were unstimulated (Unst) or stimulated with ISS-ODN, control (Cont)-ODN (5 μg/ml), probiotic (prob) DNA, methylated (m) probiotic DNA, or calf thymus (ct) DNA (20 μg/ml) for 2 hours. A) The activation of NF-kB was determined by electrophoretic mobility shift assay (EMSA). B) JNK1 activation (kinase assay). C) Cytokine levels in the supernatants were measured 24 hours post-stimulation, using an ELISA. Results are mean±SEM.

TLR Signaling is Required for Anti-Inflammatory Effects of Irradiated Probiotics

The administration of non-viable irradiated, or viable probiotics attenuated the severity of DSS induced colitis as reflected in the disease-activity score, histological score, and colonic myeloperoxidase (MPO) activity. In contrast, the administration of heat-killed probiotics had no effect on the severity of DSS induced colitis (Table 1). Recently, chloroquine has been shown to inhibit the activation of TLR9 induced by its natural ligand, bacterial DNA (Macfarlane and Manzel (1998) J Immunol 160:1122-1131). Indeed, when mice were treated with chloroquine, it completely abolished the protective effect of both viable and irradiated probiotics on experimental colitis (Table 1).

Table 1. Balb/c mice were intragastrically treated daily with 2.8×10⁸ CFU of viable, irradiated or heat-killed probiotics 10 days prior to the addition of DSS (3.5%) to the drinking water and for 7 days thereafter. Three groups were also subcutaneously treated with chloroquine (10 mg/kg) dissolved in 0.1 ml of saline once daily (see Materials and Methods). Disease activity score, colonic MPO activity and histological score were determined after 7 days of DSS administration as described. Results are mean±SEM and represent 1 of 3 experiments. The following statistical analyses were employed; for MPO activity-Student t test, for disease activity score as well as for histological score-Mann-Whitney test. *Significantly different from no treatment or chloroquine treatment (P<0.05).

TABLE 1
Effect of Probiotics and Chloroquine on DSS-Induced Colitis

| | | Disease Activity | MPO | Histological |
|---|---|---|---|---|
| Treatment | N | Score | (U/gr) | Score |
| NONE | 8 | 8.0 ± 0.9 | 1.90 ± 0.09 | 7.5 ± 0.9 |
| Viable probiotics | 8 | 2.7 ± 1.1* | 0.78 ± 0.20* | 2.8 ± 0.8* |
| Irradiated probiotics | 8 | 0.1 ± 0.1* | 1.10 ± 0.10* | 2.5 ± 0.3* |
| Heat-killed probiotics | 9 | 7.0 ± 0.8 | 1.60 ± 0.10 | 5.4 ± 1.0 |
| Chloroquine | 5 | 6.2 ± 0.4 | 1.54 ± 0.16 | 8.0 ± 0.8 |
| Irradiated probiotics + chloroquine | 5 | 6.6 ± 0.8 | 1.60 ± 0.10 | 7.7 ± 0.6 |
| Viable probiotics + chloroquine | 5 | 6.8 ± 0.1 | 2.30 ± 0.48 | 6.8 ± 0.7 |

Histologically, the extensive superficial ulceration with mucosal inflammatory reaction induced by DSS was totally abolished in mice treated with irradiated probiotics whereas in mice co-treated with viable probiotics only minimal superficial ulceration with minimal inflammatory reaction was observed.

Histological evaluation of a colonic segment of naïve Balb/c mice showed normal colonic mucosa, submucosa, and muscularis propria. Histological evaluation of a colonic segment of TLR9 null mice treated with DSS (1.75%) and irradiated probiotic bacteria showed superficial ulceration with severe acute inflammation involving mucosa, submucosa, muscularis propria, and mesenteric fat tissue. Histological evaluation of a colonic segment of Balb/c mice treated with DSS (3.5%) and viable probiotic bacteria showed minimal superficial ulceration over a lymphoid nodule along with minimal inflammatory reaction involving the mucosa only. Histological evaluation of a colonic segment of Balb/c mice treated with DSS (3.5%) and irradiated probiotic bacteria showed normal colonic mucosa, submucosa, and muscularis propria. Histological evaluation of a colonic segment of Balb/c mice following 7 days of DSS (3.5%) administration showed extensive superficial ulceration with mucosal inflammatory reaction.

Irradiated and viable probiotics as well as ISS-ODN were also found to equally attenuate the severity of a chronic model of DSS induced colitis (Table 2). In this model the probiotic preparations and the ISS-ODN were administered with or after induction of colitis, respectively, indicating their therapeutic capacity.

Table 2. Balb/c mice were treated for 7 days with DSS (3.5%) added to the drinking water and for an additional 7 days with DSS (1.75%). One group was treated on day 8 s.c. with ISS-ODN (10 μg) and two other groups were treated daily i.g. with viable or irradiated probiotics 2.8×10⁸ CFU. Mice were sacrificed on day 15. Results are mean±SEM and represent 1 of 3 experiments. For MPO activity, Student t test was employed. For disease activity score and for histological score, Mann-Whitney test was employed *Significantly different from no treatment (P<0.05).

TABLE 2
Effect of Probiotics on Chronic DSS-Induced Colitis

| | | Disease Activity | MPO | Histological |
|---|---|---|---|---|
| Treatment | N | Score | (U/gr) | Score |
| NONE | 9 | 5.4 ± 0.6 | 1.60 ± 0.10 | 8.0 ± 0.5 |
| Viable probiotics | 7 | 0.9 ± 0.5* | 0.97 ± 0.10* | 6.5 ± 0.7 |
| Irradiated probiotics | 8 | 1.6 ± 0.5* | 1.18 ± 0.10* | 5.9 ± 0.1* |
| ISS-ODN | 8 | 1.1 ± 0.4* | 1.25 ± 0.19* | 6.9 ± 0.8 |

Probiotic and E. coli DNA Inhibit DSS-Induced Colitis

To evaluate the anti-inflammatory role of probiotic DNA in experimental colitis, probiotic DNA was delivered i.g., i.r. (Table 3) or s.c. (Table 4) once, two hrs prior to DSS administration. Intragastric and s.c administration of probiotic DNA or ISS-ODN inhibited the severity of DSS-induced colitis whereas i.r. administration of these compounds had no effect on the outcome of colitis. The i.g. administration of methylated probiotic DNA (i.e., with CpG methylase), calf thymus DNA, or DNase treated probiotics (i.e., the amount of microorganisms that yielded 50 μg of probiotic DNA) also did not affect the course or the severity of colitis (Table 3). Intragastric or s.c. administration of E. coli DNA also inhibited the severity of DSS-induced colitis (Table 5). Taken together, these data outline the anti-inflammatory role of certain microbial DNA and the required i.g. or s.c. route of administration for the attenuation of experimental colitis.

Table 3. Balb/c mice were intragastrically or intrarectally treated with various DNA preparations 2 hours before induction of colitis with DSS. Results are mean±SEM and represent 1 of 3 experiments. For MPO activity, Student t test was employed. For disease activity score and for histological score, Mann-Whitney test was employed. *Significantly different from no treatment or treatment with calf thymus DNA (P<0.05). ** Significantly different from DNase treated probiotics (P<0.05).

TABLE 3
Effect of Intragastric or Intrarectal Administration of
Various Probiotic DNA(s) on DSS-Induced Colitis

| | | Disease Activity | MPO | Histological |
|---|---|---|---|---|
| Treatment | N | Score | (U/gr) | Score |
| NONE | 19 | 7.3 ± 0.5 | 1.90 ± 0.09 | 7.5 ± 0.9 |
| Probiotic DNA (i.g.) | 8 | 3.2 ± 0.7*^{;}** | 1.30 ± 0.10*^{;}** | 3.0 ± 0.4* |
| Probiotic DNA (i.r.) | 10 | 5.9 ± 1.2 | 1.90 ± 0.10 | 6.5 ± 1.1 |
| Methylated Probiotic DNA (i.g.) | 4 | 7.8 ± 0.5 | 1.93 ± 0.35 | 5.7 ± 0.7 |
| DNase treated Probiotics (i.g.) | 6 | 7.8 ± 1.2 | 1.60 ± 0.20 | 7.4 ± 1.4 |
| Calf thymus DNA (i.g.) | 8 | 5.7 ± 1.0 | 1.98 ± 0.20 | 6.3 ± 1.4 |
| ISS-ODN (i.g.) | 8 | 3.6 ± 0.7* | 1.09 ± 0.10* | 2.8 ± 0.6* |
| ISS-ODN (i.r.) | 10 | 6.3 ± 0.7 | 1.90 ± 0.30 | 5.9 ± 0.7 |
| Control-ODN (i.g.) | 10 | 6.7 ± 0.8 | 1.90 ± 0.20 | 5.7 ± 0.3 |

Table 4. Balb/c mice were subcutaneously injected with various DNA preparations 2 hours before induction of colitis (see Materials and Methods). Results are mean±SEM and represent 1 of 3 experiments. For MPO activity, Student t test was employed. For disease activity score and for histological score, Mann-Whitney test was employed. * Significantly different from no treatment (P<0.05).** Significantly different from treatment with probiotic DNA (P<0.05).

TABLE 4
Effect of Subcutaneous Administration of Various
DNA(s) on DSS-Induced Colitis

| | | Disease | MPO | Histological |
|---|---|---|---|---|
| Treatment | N | Activity Score | (U/gr) | Score |
| NONE | 10 | 5.7 ± 0.5 | 3.6 ± 0.3 | 10.7 ± 0.8 |
| Probiotic DNA | 8 | 1.1 ± 0.3* | 0.8 ± 0.1* | 0.3 ± 0.3* |
| Methylated Probiotic | 8 | 3.4 ± 0.4** | 1.7 ± 0.2** | 4.3 ± 1.3** |
| DNA | | | | |
| DNase treated | 4 | 5.0 ± 1.1** | 1.5 ± 0.2** | 4.3 ± 1.2** |
| Probiotics | | | | |
| Calf thymus DNA | 4 | 5.5 ± 1.5 | 3.3 ± 0.5 | 6.0 ± 1.3 |
| ISS-ODN | 4 | 0.4 ± 0.4* | 0.9 ± 0.1* | 0 ± 0* |
| Control-ODN | 4 | 3.8 ± 0.7 | 4.2 ± 0.8 | 8.0 ± 0.9 |

Table 5. Balb/c mice were subcutaneously or intragastrically treated with E. coli DNA 2 hours before induction of colitis DSS (see Materials and Methods). Results are mean±SEM and represent 1 experiment. For MPO activity, Student t test was employed. For disease activity score and for histological score, Mann-Whitney test was employed. *Significantly different from no treatment (P<0.01); ** Significantly different from no treatment (P<0.03)

TABLE 5
Effects of Administration of E. coli DNA on DSS-Induced Colitis

| | | Disease | MPO | Histological |
|---|---|---|---|---|
| Treatment | N | Activity Score | (U/gr) | Score |
| NONE | 7 | 3.0 ± 0.8 | 1.40 ± 0.20 | 6.70 ± 0.7 |
| E. coli DNA (s.c.) | 7 | 0.3 ± 0.2* | 0.55 ± 0.10* | 3.10 ± 0.6** |
| E. coli DNA (i.g.) | 7 | 0.6 ± 0.3* | 0.70 ± 0.19** | 6.40 ± 0.9 |

Probiotic DNA Attenuates Different Models of Experimental Colitis

We further evaluated whether the protective effect of probiotic DNA can be reproduced by its s.c. injection in TNBS-induced colitis as well as in spontaneous colitis observed in IL-10 deficient mice (Strober et al. (2002) Annu Rev Immunol 20:495-549). A single s.c. injection of probiotic DNA, but not of methylated probiotic DNA or of calf thymus DNA, decreased the disease-activity score, histological score, and colonic MPO activity of TNBS-induced colitis (Table 6) and attenuated the course and the severity of the colitis that had developed in IL-10 deficient mice over time (Table 7).

Table 6. Balb/c mice were subcutaneously injected with various DNA preparations 2 hours before induction of colitis (see Materials and Methods). Results are mean±SEM and represent 1 of 3 experiments. For MPO activity, Student t test was employed. For disease activity score and for histological score, Mann-Whitney test was employed. *Significantly different from no treatment or treatment with calf thymus DNA (P<0.05). ** Significantly different from treatment with probiotic DNA (P<0.05)

TABLE 6
Effect of Various DNAs on TNBS-Induced Colitis

| | | Disease | MPO | Histological |
|---|---|---|---|---|
| Treatment | N | Activity Score | (U/gr) | Score |
| NONE | 10 | 1.8 ± 0.7 | 1.50 ± 0.01 | 9.0 ± 1.8 |
| Probiotic DNA | 8 | 0* | 0.88 ± 0.08* | 1.7 ± 0.9* |
| Methylated | 8 | 1.5 ± 0.3** | 1.50 ± 0.09** | 5.0 ± 0.9** |
| Probiotic DNA | | | | |
| DNase treated | 8 | 1.0 ± 0** | 1.60 ± 0.06** | 5.5 ± 0.9** |
| Probiotics | | | | |
| Calf thymus DNA | 8 | 3.4 ± 0.5 | 1.20 ± 0.07 | 7.0 ± 2.0 |
| ISS-ODN | 8 | 0.4 ± 0.3* | 0.8 ± 0.10* | 0 ± 0* |
| Control-ODN | 4 | 1.3 ± 0.2 | 2.00 ± 0.30 | 4.8 ± 1.8 |

Table 7. IL-10 KO mice (B6) were subcutaneously injected once a week with various DNAs (see Materials and Methods). The following statistical analyses were employed; for rectal prolapse-Chi square, for MPO activity-Student t test and for histological score-Mann-Whitney test. Results are mean±SEM. *Significantly different from no treatment (P<0.05).

TABLE 7
Effect of Various DNAs on Spontaneous
Colitis in IL-10 KO Mice

| | | | MPO | Histological |
|---|---|---|---|---|
| Treatment | N | Rectal Prolapse (N) | (U/gr) | Score |
| NONE | 13 | 11 | 1.0 ± 0.10 | 8.1 ± 0.9 |
| Probiotic DNA | 10 | 2* | 0.2 ± 0.04* | 3.0 ± 0.4* |
| Calf Thymus | 8 | 5 | 0.7 ± 0.10 | 6.3 ± 1.4 |
| DNA | | | | |
| ISS-ODN | 6 | 1* | 0.8 ± 0.10 | 1.8 ± 1.2* |

Absorption of Bacterial DNA from the Gastrointestinal Tract

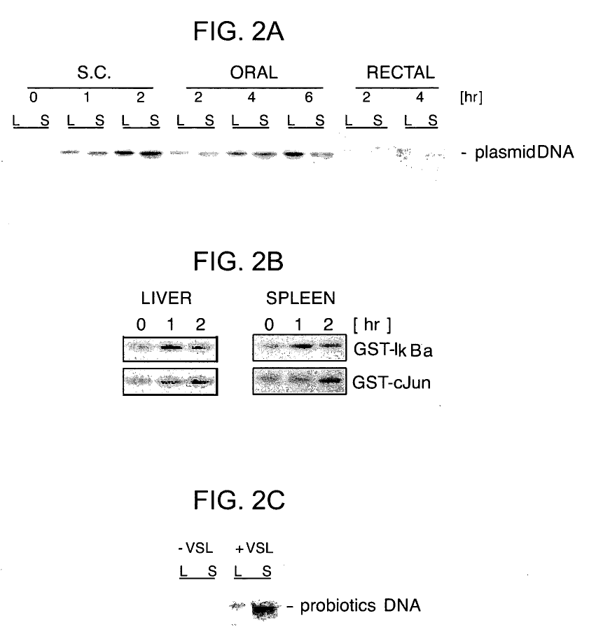

As i.g. or s.c. but not i.r. administration of probiotic DNA ameliorates experimental colitis, we reasoned that the probiotic DNA might be absorbed from the upper gastrointestinal tract as was described for phage DNA (Schubbert et al. (1997) Proc Natl Acad Sci USA 94:961-966) and act in systemic sites. To explore this possibility, a purified form of bacterial DNA was delivered, i.e., plasmid DNA (pDNA), i.g. once to wt mice and the presence of this bacterial DNA in their liver and spleen was evaluated. Indeed, we identified the pDNA and its fragments in these organs (southern blot) within 2-6 hrs post-i.g. but not post-i.r. administration (FIG. 2A). Interestingly, the efficacy of bacterial DNA absorption was by far lower when the pDNA was delivered i.r rather than i.g. (FIG. 2A). The localization of this bacterial DNA in these organs coincided with its immunostimulatory activities i.e., the activation of JNK and NF-kB (FIG. 2B), the major signaling pathways initiated by the engagement of TLR9 with its ligand, bacterial DNA. We also identified the probiotic DNA in the liver and spleen after daily i.g. administration of irradiated probiotics which was initiated 10 days prior to induction of colitis with DSS, and for 7 days thereafter (FIG. 2C).

FIGS. 2A-C: Detection of bacterial DNA at systemic sites. A) Plasmid DNA is detected after its oral administration (1 mg/mouse) in the liver (L) and spleen (S) but is not detected after rectal administration (southern blot). The uptake of pDNA in these organs after s.c. injection (100 μg/mouse) is shown as control. B) The localization of orally administered pDNA coincides with the activation of IKK and JNK1 in these organs. C) VSL DNA is detected in the liver (L) and the spleen (S) after 17 days of oral administration of irradiated probiotic bacteria (southern blot).

Taken together, these data indicate that most of the probiotic DNA is absorbed from the upper gastrointestinal tract and most probably acts systemically as occurs with s.c. injection of other types of immunostimulatory DNA (e.g., ISS-ODN).

### Example 2

### Administration of Pasteurized Probiotics Ameliorates DSS Induced Colitis

Live bacteria from the VSL-3 preparation were heated at different temperatures for various time periods as indicated. Following the heating, the bacteria were cultured for 48 hours and counted. 2.8×10⁸ heated bacteria were administered i.g. to Balb/c mice for 10 days prior to the additions of DSS 5% to their drinking water and for 7 days thereafter. Seven days after the addition of DSS, mice were sacrificed disease activity (DAI; % decrease in body weight and rectal bleeding) and MPO activity were determined and sections were obtained for histological analysis (HS; histological score). The results are shown in Table 8, below. DAI=disease activity score (index).

TABLE 8

| Treatment | Time | Culture | N | DAI | MPO |
|---|---|---|---|---|---|
| 63° C. | 30 min. | 10⁶ | 9 | 3.3 ± 0.7 | 1.1 ± 0.20 |
| 70° C. | 5 min. | 10⁶ | 10 | 3.1 ± 0.8 | 1.50 ± 0.20 |
| 80° C. | 10 min. | sterile | 15 | 4.7 ± 0.5 | 1.95 ± 0.20 |
| 100° C. | 30 min. | sterile | 10 | 7.3 ± 0.5 | 1.98 ± 0.20 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An enteral formulation comprising inactivated probiotic bacteria; and a pharmaceutically acceptable excipient, wherein the bacteria are inactivated by a process other than heating to 100° C. for 30 minutes.

2. The method of claim 1, wherein the bacteria are inactivated by a process selected from gamma irradiation, ultraviolet irradiation, and pasteurization.

3. The formulation of claim 1, wherein the formulation is a liquid or gel formulation comprising an agent selected from the group of a flavoring agent and a coloring agent.

4. The formulation of claim 1, wherein the formulation is a solid formulation comprising a solid-based dry material.

5. The formulation of claim 4, wherein the solid-based dry material is selected from a starch, gelatin, sucrose, dextrose, trehalose, and malto-dextrin.

6. The formulation of claim 1, wherein said formulation is in the form of a capsule, tablet, a liquid, or a gel.

7. The formulation of claim 1, wherein said pharmaceutically acceptable excipient is a food-grade carrier.

8. The formulation of claim 7, wherein the food-grade carrier is selected from an edible oil, an emulsifier, a soluble fiber, a flavoring agent, a coloring agent, an edible fiber, and a sweetener.

9. The formulation of claim 1, wherein the inactivated bacteria are present at a concentration of from about 1×10⁵ bacteria per gram to about 1×10¹⁴ bacteria per gram.

10. The formulation of claim 1, wherein the inactivated bacteria are present in a concentration of least about 5% by weight.

11. The formulation of claim 1, wherein the formulation is a liquid formulation, and the inactivated bacteria are present at a concentration of from about 1×10⁵ bacteria per milliliter to about 1×10¹⁴ bacteria per milliliter.

12. The formulation of claim 1, further comprising an immunosuppressive agent.

13. The formulation of claim 12, wherein the immunosuppressive agent is selected from a steroidal agent, azathioprine, 6-mercaptopurine, methotrexate, cyclosporine, tacrolimus, mycophenolate mofetil, and thalidomide.

14. The formulation of claim 1, further comprising an antibiotic.

15. The formulation of claim 14, wherein the antibiotic is selected from metonidazole and ciprofloxacin.

16. The formulation of claim 1, further comprising sulfasalazine.

17. The formulation of claim 1, further comprising 5-aminosalicylic acid.

18. The formulation of claim 1, further comprising a nutritional beverage comprising nutrients that are readily absorbed by gut epithelium.

19. A food product comprising inactivated probiotic bacteria, wherein the bacteria are inactivated by a process other than heating to 100° C. for 30 minutes, and wherein the inactivated bacteria are present in the food product at a concentration of from about 1×10⁵ bacteria per gram to about 1×10¹⁴ bacteria per gram or from about 1×10⁵ bacteria per milliliter to about 1×10¹⁴ bacteria per milliliter.

20. The food product of claim 19, wherein the food product is a milk-based food product.

21. The food product of claim 19, wherein the milk-based food product is selected from milk, cheese, yogurt, butter, ice cream, frozen yogurt, whipped toppings, cream, custard, pudding, nutritional drinks, infant formula, and milk chocolate.

22. The food product of claim 19, wherein the food product is a soy-based food product.

23. A method of treating a disorder that is amenable to treatment with viable probiotic bacteria, the method comprising administering to an individual in need thereof an effective amount of a formulation of claim 1.

24. The method of claim 23, wherein the formulation is administered orally.

25. The method of claim 23, wherein the disorder is gastrointestinal inflammation.

26. The method of claim 25, wherein the gastrointestinal inflammation is acute gastrointestinal inflammation.

27. The method of claim 25, wherein the gastrointestinal inflammation is chronic gastrointestinal inflammation.

28. The method of claim 27, wherein the chronic gastrointestinal inflammation is caused by inflammatory bowel disease.

29. The method of claim 28, wherein the inflammatory bowel disease is ulcerative colitis.

30. The method of claim 28, wherein the inflammatory bowel disease is Crohn disease.

31. The method of claim 23, wherein from about 1×10⁵ bacteria per gram to about 1×10¹⁴ bacteria per unit dosage form are administered.

32. The method of claim 23, wherein the disorder is an allergic disorder.

33. The method of claim 32, further comprising administering an additional therapeutic agent for treating the allergic disorder.

34. The method of claim 32, wherein the allergic disorder is allergic asthma.

35. The method of claim 32, wherein the allergic disorder is an allergic reaction to a plant allergen, a food allergen, an animal allergen, or a drug allergen.

36. The method of claim 32, wherein the allergic disorder is selected from atopic dermatitis, a food allergy, allergic gastroenteritis, and allergic rhinitis.

37. The method of claim 23, wherein the disorder is a diarrheal disease.

38. The method of claim 37, wherein the diarrheal disease is caused by a bacterial infection, a viral infection, a mixed viral and bacterial infection, radiation treatment, or antibiotic treatment.

39. The method of claim 23, wherein the disorder is irritable bowel syndrome.

40. The method of claim 23, wherein the disorder is non-alcoholic liver disease.
